(19) United States
(12) Patent Application Publication
(10) Pub. No.: US 2022/0211836 A1
(43) Pub. Date: Jul. 7, 2022

(54) INACTIVATED VIRUS COMPOSITIONS AND ZIKA VACCINE FORMULATIONS

(71) Applicant: Takeda Vaccines, Inc., Cambridge, MA (US)

(72) Inventors: Michael JOHNSON, Cambridge, MA (US); Sushma KOMMAREDDY, Cambridge, MA (US)

(73) Assignee: Takeda Vaccines, Inc., Cambridge, MA (US)

(21) Appl. No.: 17/609,217

(22) PCT Filed: Apr. 8, 2020
(86) PCT No.: PCT/US2020/027154

§ 371 Date: Nov. 5, 2021

Related U.S. Application Data
(60) Provisional application No. 62/845,024, filed on May 8, 2019.

(51) Int. Cl. A61K 39/12; A61K 47/10; A61K 47/26; A61K 47/02

(52) U.S. Cl. CPC A61K 39/12; A61K 47/10; A61K 2039/5252; A61K 47/02; A61K 47/26

(57) ABSTRACT

The present invention relates to a liquid inactivated virus composition comprising: an inactivated whole Zika virus, at least one pharmaceutically acceptable buffer with a concentration of at least about 6.5 mM, and optionally a polyol, wherein said at least one pharmaceutically acceptable buffer does not comprise phosphate ions and vaccines derived therefrom.

### TECHNICAL FIELD OF THE INVENTION

The present disclosure relates to inactivated virus compositions comprising an inactivated whole Zika virus and formulation, methods of manufacture, and uses thereof as well as vaccines derived therefrom.

### BACKGROUND OF THE INVENTION

Zika virus, a flavivirus classified with other mosquito-borne viruses (e.g., yellow fever, dengue, West Nile, and Japanese encephalitis viruses) within the Flaviviridae family has spread rapidly in a hemispheric-wide epidemic since the virus was introduced into Brazil in 2013. The virus has reached the Central and North Americas, including territories of the United States, consequently now threatening the continental US. Indeed, Zika virus strain PRVABC59 was isolated from serum from a person who had travelled to Puerto Rico in 2015. The genome of this strain has been sequenced at least three times (See Lanciotti et al. Emerg. Infect. Dis. 2016 May; 22(5):933-5 and GenBank Accession Number KU501215.1; GenBank Accession Number KX087101.3; and Yun et al. Genome Announc. 2016 Aug. 18; 4(4) and GenBank Accession Number ANK57897.1).

Initially isolated in 1947 in Uganda, the virus was first linked to human disease in 1952, and has been recognized sporadically as a cause of mild, self-limited febrile illness in Africa and Southeast Asia (Weaver et al. (2016) Antiviral Res. 130:69-80; Faria et al. (2016) Science. 352(6283):345-349). However, in 2007, an outbreak appeared in the North Pacific island of Yap, and then disseminated from island to island across the Pacific, leading to an extensive outbreak in 2013-2014 in French Polynesia, spreading then to New Caledonia, the Cook Islands, and ultimately, to Easter Island. An Asian lineage virus was subsequently transferred to the Western Hemisphere by routes that remain undetermined (Faria et al. (2016) Science. 352(6283):345-349). The virus may be transmitted zoonotically by Aedes aegypti, A. albopictus, and possibly by A. hensilli and A. polynieseinsis (Weaver et al. (2016) Antiviral Res. 130:69-80). Additionally, it is thought that other vectors for transmitting the virus may exist, and the virus may be transmitted by blood transfusion, transplacentally, and/or through sexual transmission.

In late 2015, a significant increase in fetal abnormalities (e.g., microcephaly) and Guillain-Barre syndrome (GBS) in areas of widespread Zika virus infection raised alarm that Zika virus might be much more virulent than originally thought, prompting the World Health Organization (WHO) to declare a Public Health Emergency of International Concern (PHEIC) (Heymann et al. (2016) Lancet 387(10020): 719-21). While Zika virus poses a substantial public health threat, no FDA-approved vaccine or treatment currently exists, and the only preventative measures for controlling Zika virus involve managing mosquito populations.

In recent efforts to characterize a recombinant Zika virus for the development of a potential vaccine, a non-human cell adapted Zika virus was identified that harbors a mutation in the viral Envelope protein at position 330 (Weger-Lucarelli et al. 2017. Journal of Virology). The authors of this study found that full-length infectious cDNA clones of Zika virus strain PRVABC59 were genetically unstable when amplified during cloning, and opted to split the viral genome to address the observed instability, developing and applying a two plasmid system. However, a two plasmid system for the development of a Zika vaccine is less desirable. Thus, there is a need to develop vaccines for treating and/or preventing Zika virus infection that utilize a genetically stable Zika virus.

It is desirable that inactivated virus compositions demonstrate good stability, in particular it is necessary during the manufacture of vaccines that inactivated virus compositions as intermediate products usually referred to as “drug substance” which is already purified and inactivated can be transported and stored for extended periods of time and not lose their activity when waiting for being formulated to the end product, i.e. the vaccine. For example inactivated virus compositions may be frozen (such as e.g. at −80° C.), so that they can be stored for extended periods of time. Consequently, it is important that inactivated virus compositions are stable during storage at −80° C. Furthermore, it is important that inactivated virus compositions are able to withstand changes in temperature. In particular, it is important that inactivated virus compositions do not lose activity as a result of freezing and thawing, which results in one or multiple freeze thaw cycles. Resistance to one or multiple freeze thaw cycles also means that it is possible to freeze drug substance material during the production process.

### OBJECTS AND SUMMARY OF THE INVENTION

It is an object of the present invention to provide inactivated virus compositions in which an inactivated whole Zika virus is stabilized during storage. In particular, it is an object of the present invention to provide inactivated virus compositions in which an inactivated whole Zika virus is stabilized during storage at −80° C. for extended periods of time, such as e.g. storage for at least 10 days until e.g. 6 months or a year. Such compositions intended to be stored frozen in general do not (yet) contain an aluminium-based adjuvant such as an aluminum salt such as alum/aluminum hydroxide—if so desired, such aluminium-based adjuvants can be added later on, when no more frozen storage is scheduled.

It is a further object of the present invention to provide inactivated virus compositions that are able to stabilize an inactivated whole Zika virus during one or multiple freeze thaw cycles.

The above objects are achieved by embodiments of the present invention as described and claimed herein.

The present invention is, therefore, directed to a liquid inactivated virus composition comprising:
a) an inactivated whole Zika virus,
b) at least one pharmaceutically acceptable buffer with a concentration of at least about 6.5 mM, and
c) optionally a polyol,
   wherein the liquid inactivated virus composition preferably does not contain an adjuvant selected from aluminum salts, and
   said at least one pharmaceutically acceptable buffer does not comprise phosphate ions.

In a certain aspect, the concentration of phosphate ions within the liquid inactivated virus composition is less than about 7 mM, or less than about 6 mM, or less than about 5 mM, or less than about 4 mM, or less than about 3 mM, or less than about 2 mM, or less than about 1 mM.

The present invention is also directed to the use of an inactivated virus composition comprising:
a) an inactivated whole Zika virus,
b) at least one pharmaceutically acceptable buffer with a concentration of at least about 6.5 mM, and
c) optionally a polyol,
   wherein the liquid inactivated virus composition preferably does not contain an adjuvant selected from aluminum salts and
   wherein said at least one pharmaceutically acceptable buffer does not comprise phosphate ions, for stabilizing the inactivated whole Zika virus.

The present invention is further directed to a method of preparing a liquid inactivated virus composition comprising:
a) an inactivated whole Zika virus,
b) a pharmaceutically acceptable buffer, wherein the said buffer is not phosphate buffer and wherein the concentration of said buffer is at least 6.5 mM; and
c) optionally a polyol,
   wherein the inactivated virus composition does not contain an adjuvant selected from aluminum salts, the method comprising the following steps:
Step 1. isolating a Zika virus preparation from supernatants obtained from one or more non-human cells;
Step 2. purifying the Zika virus preparation;
Step 3. inactivating the virus preparation;
Step 4. transferring the Zika virus preparation into a pharmaceutically acceptable buffer to obtain the Zika virus drug substance.

The present invention is further directed to a liquid vaccine comprising:
a) the inactivated virus composition in accordance with the invention, and
b) an adjuvant such as aluminum hydroxide.

In a certain aspect, the liquid vaccine comprises from about 50 mM to about 200 mM NaCl, from about 8.5 mM to about 80 mM Tris and from about 0.4% w/v to about 4.7% w/v sucrose.

The present invention is also directed to a method of treating or preventing, in particular preventing a Zika virus infection in a human subject in need thereof, comprising administering to the subject a unit dose of the liquid vaccine in accordance with the present invention, as described above.

The present invention is further directed to a method of preparing a liquid vaccine, the method comprising the following steps:
Step 1. providing the inactivated virus composition according to the present invention, as described above,
Step 2. adding an adjuvant, the adjuvant preferably being an aluminum salt, and optionally a further pharmaceutically acceptable buffered liquid to the inactivated virus composition.

The present invention is also directed to a product obtainable by the method described above.

### BRIEF DESCRIPTION OF THE DRAWINGS

FIG. 1 shows bright field microscopy images of Vero cell monolayers mock infected (top) or infected with ZIKAV strain PRVABC59 (bottom).

FIG. 2 shows growth kinetics of ZIKAV PRVABC59 P1 on Vero cell monolayers, as determined by TCID₅₀.

FIG. 3 shows potency assay testing (TCID₅₀) of Zika virus PRVABC59 P5 clones a-f.

FIG. 4 shows bright-field microscopy images depicting the cytopathic effect (CPE) of growth of Zika virus PRVABC59 P6 clones a-f on Vero cell monolayers.

FIG. 5 shows potency assay testing (TCID₅₀) of Zika virus PRVABC59 P6 clones a-f

FIG. 6 shows an amino acid sequence alignment comparing the envelope glycoprotein sequence of Zika virus near residue 330 from Zika virus strains PRVABC59 P6e (SEQ ID NO: 8) and PRVABC59 (SEQ ID NO: 9) with several other flaviviruses (WNV (SEQ ID NO: 10); JEV (SEQ ID NO: 11); SLEV (SEQ ID NO: 12); YFV (SEQ ID NO: 13); DENV 1 16007 (SEQ ID NO: 14); DENV 2 16681 (SEQ ID NO: 15); DENV 3 16562 (SEQ ID NO: 16); and DENV 4 1036 (SEQ ID NO: 17)).

FIG. 7 shows an amino acid sequence alignment comparing the NS1 protein sequence of Zika virus near residue 98 from Zika virus strains PRVABC59 P6e (SEQ ID NO: 18) and PRVABC59 (SEQ ID NO: 19) with several other flaviviruses (WNV (SEQ ID NO: 20); JEV (SEQ ID NO: 21), SLEV (SEQ ID NO: 22); YFV (SEQ ID NO: 23); DENV 1 16007 (SEQ ID NO: 24); DENV 2 16681 (SEQ ID NO: 25); DENV 3 16562 (SEQ ID NO: 26); and DENV 4 1036 (SEQ ID NO: 27)).

FIG. 8 shows the plaque phenotype of ZIKAV PRVABC59 P6 virus clones a-f compared to ZIKAV PRVABC59 P1 virus.

FIG. 9 shows the mean plaque size of ZIKAV PRVABC59 P6 virus clones compared to ZIKAV PRVABC59 P1 virus.

FIG. 10 shows the growth kinetics of ZIKAV PRVABC59 P6 clones a-f in Vero cells under serum-free growth conditions.

FIG. 11 shows compiled kinetics of inactivation data. Data compares infectious potency (TCID50) to RNA copy, and completeness of inactivation (COI) for samples from the four toxicology lots. These data indicate that the sensitivity of the COI assay is greater than TCID50.

FIG. 12 shows a comparison of C6/36 and Vero sensitivity in the assay as demonstrated with an input virus titer of 0.31 TCID50.

FIG. 13 shows a logistic regression analysis of CPE vs. log TCID50 using C6/36 cells site that include 99% confidence intervals around a target value of 0.01 TCID50/well (−2 log TCID50/well); the model predicts 0.85% of wells will be positive.

FIG. 14: The peak corresponding to the intact Zika virus (retention time ca. 8 minutes) in the SEC chromatogram for the Zika virus vaccine drug substance in Tris+7% sucrose buffer, following storage at −80° C. for 67 days (this peak corresponds to example 3C, Table 16b).

FIG. 15: The peak corresponding to the intact Zika virus (retention time ca. 8 minutes) in the SEC chromatogram for the Zika virus vaccine drug substance in ZPB buffer, following storage at −80° C. for 67 days (this peak corresponds to example 3C, Table 16b).

FIG. 16: The percentage of intact Zika virus remaining (measured by SEC) after 10 days of storage at 5±3° C. and −80° C. (corresponding to example 3A).

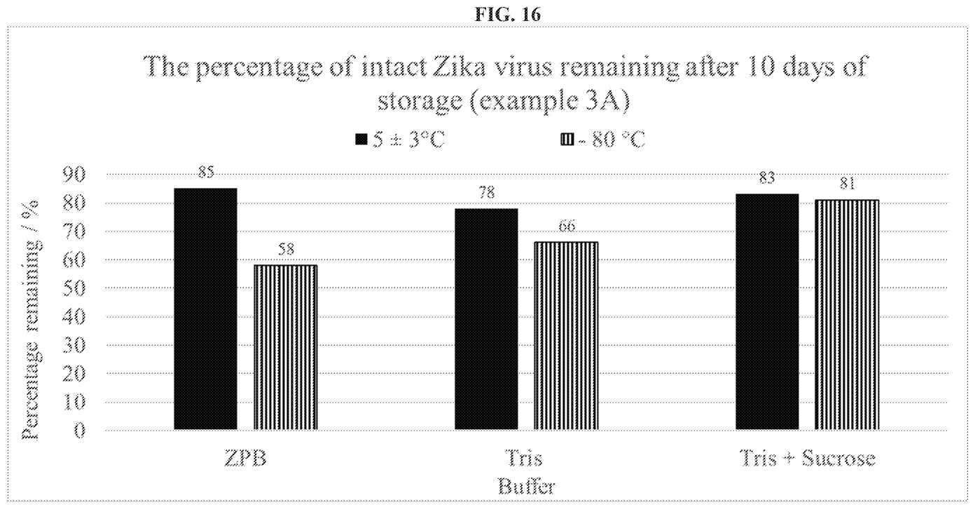

FIG. 17: The percentage of intact Zika virus remaining (measured by SEC) after 60 days of storage at −80° C. (corresponding to example 3B).

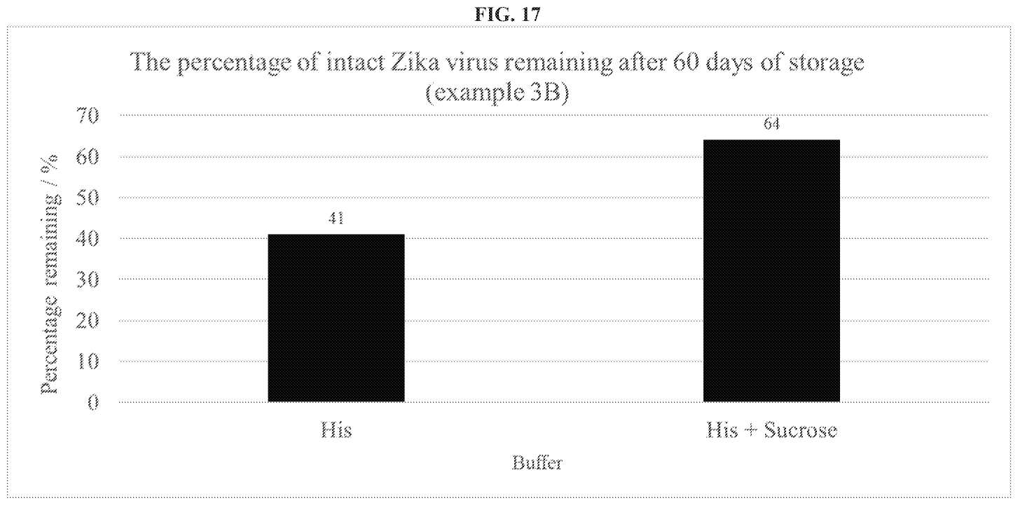

FIG. 18: The percentage of intact Zika virus remaining (measured by SEC) after 67 days of storage at 5±3° C. and −80° C. (corresponding to example 3C).

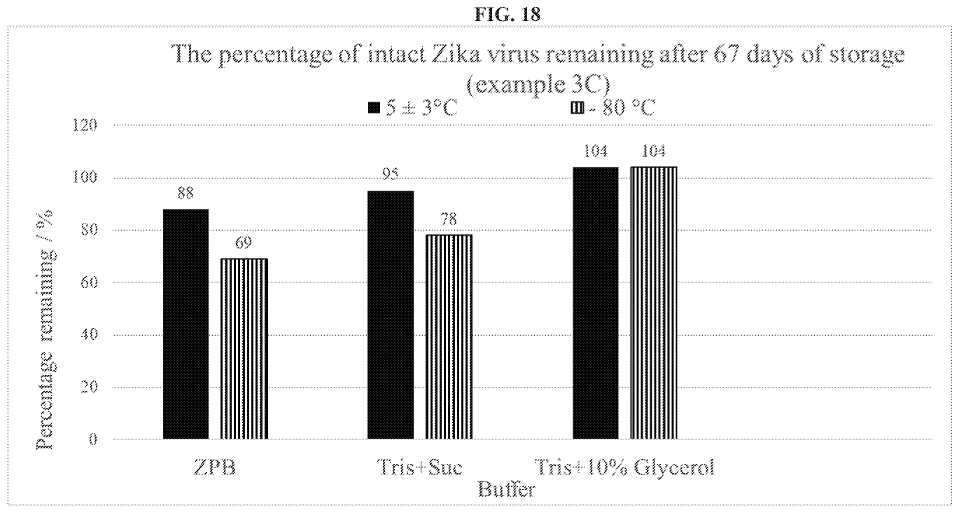

FIG. 19: The percentage of intact Zika virus remaining (measured by SEC) for Zika vaccine drug substance in ZPB and Tris+Suc, after storage for 3 months at −80° C. (corresponding to example 3D).

FIG. 20: The percentage of intact Zika virus remaining (measured by SEC) for Zika vaccine drug substance in ZPB and TBS, after storage for 0 to 60 days at 5 t 3° C. (corresponding to example 3E).

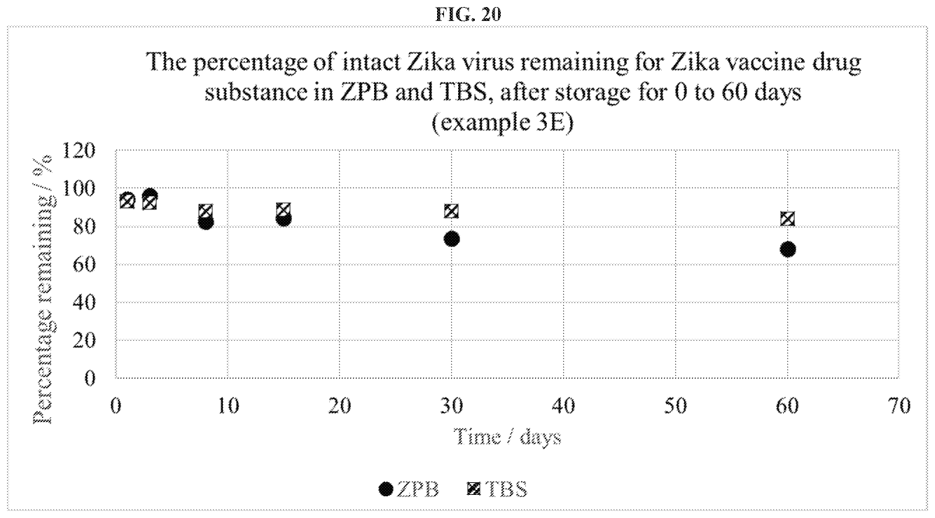

FIG. 21: The percentage of intact Zika virus remaining (measured by SEC) after repeated freeze-thaw cycles (corresponding to example 3F).

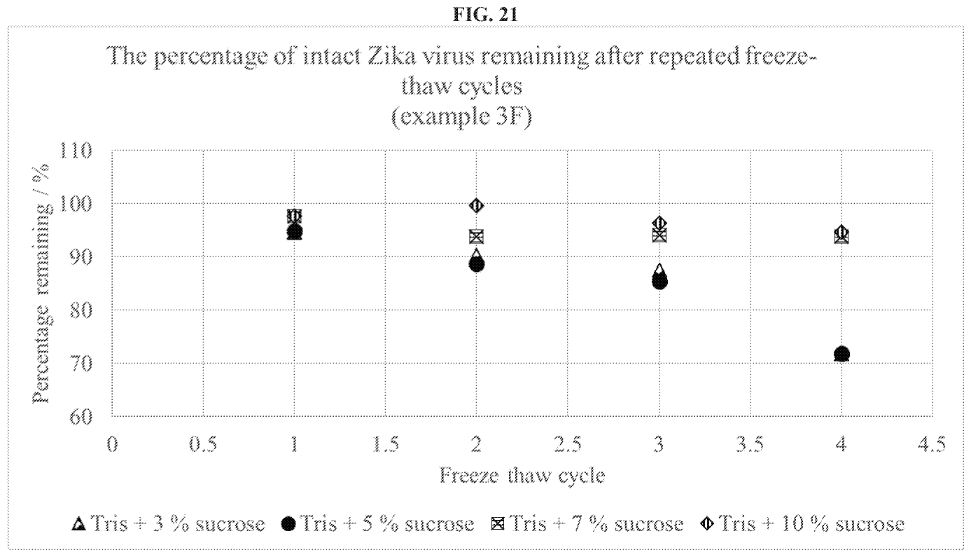

### DEFINITIONS

Unless defined otherwise, all technical and scientific terms used herein have the same meaning as commonly understood by one of ordinary skill in the art to which the invention pertains. Although, any methods and materials similar or equivalent to those described herein can be used in practice for testing of the present invention, the preferred materials and methods are described herein. In describing and claiming the present invention, the following terminology will be used in accordance with the definitions set out below.

Unless clearly indicated otherwise, use of the terms “a,” “an,” and the like refers to one or more.

The term “inactivated Zika virus” as used herein is intended to comprise a Zika virus, which has been treated with an inactivating method such as treatment with an effective amount of fomaldehyde.

The term “inactivated whole Zika virus” as used herein is intended to comprise a Zika virus, which has been treated with an inactivating method such as treatment with an effective amount of formalin. Such a treatment is considered not to destroy the structure of the virus, i.e. it does not destroy the secondary, tertiary or quaternary structure and immunogenic epitopes of the virus, but the inactivated Zika virus is no longer able to infect host cells, which can be infected with a Zika virus that has not been inactivated. In one embodiment, the inactivated Zika virus is no longer able to infect VERO cells and exert a cytopathic effect on the VERO cells. In particular, the inactivated (whole) Zika virus may be obtainable/obtained from a method wherein the Zika virus is treated with formaldehyde in an amount of about 0.01% w/v for 10 days at a temperature of 20° C. to 24° C. A sample of whole Zika virus may provide a main peak of at least 85% of the total area under the curve in the size exclusion chromatography.

The term “polyol” is defined for purposes of the present invention to refer to a substance with multiple hydroxyl groups, and includes sugars (reducing and non-reducing sugars), sugar alcohols and sugar acids. Another example for a polyol is glycerol. Optionally, polyols as defined herein have a molecular weight which is less than about 600 Da (e.g. in the range from about 120 to about 400 Da).

The term “amino group containing molecule” is defined for the purposes of the present invention to include primary, secondary, tertiary and quaternary amine group (RNH₂, R₂NH, R₃N, R₄N+) containing molecules. The R group is generally a cyclic or acyclic hydrocarbon. Amino group containing molecules include Amino acids (such as e.g. histidine), Tris, ACES, CHES, CAPSO, TAPS, CAPS, Bis-Tris, TAPSO, TES, Tricine, and ADA (all of the abbreviations for each of the buffers has the same meaning as generally known in the art).

The term “room temperature” is defined for purposes of the present invention to refer to normal room temperature, such as e.g. about 25° C.

Within the meaning of the present invention, the terms “inactivated virus composition” or “liquid inactivated virus composition” generally refer to a composition in liquid form or in the form of a frozen liquid. Such compositions are intermediate compositions including the inactivated virus which are often stored in a frozen state and then are used to finally prepare the vaccine/drug product by at least further adding adjuvants.

“Tris” refers to tris(hydroxymethyl)aminomethane buffer.

If not indicated otherwise “%” refers to “weight per volume (w/v)”

### DETAILED DESCRIPTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); and The Antibodies (M. Zanetti and J. D. Capra, eds., Harwood Academic Publishers, 1995).

### Inactivated Virus Composition

The present invention is directed to a liquid inactivated virus composition comprising:
a) an inactivated whole Zika virus,
b) at least one pharmaceutically acceptable buffer with a concentration of at least about 6.5 mM, and
c) optionally a polyol,
   wherein said at least one pharmaceutically acceptable buffer does not comprise phosphate ions.

In certain such embodiments, the liquid inactivated virus composition does not contain an adjuvant selected from aluminum salts. In particular, the aluminum salts may be selected from the group of alum (such as aluminum hydroxide), aluminum phosphate, aluminum hydroxide, potassium aluminum sulfate. In certain such embodiments, the liquid inactivated virus composition does not contain adjuvants to which the inactivated whole Zika virus can be absorbed to. In certain such embodiments, the liquid inactivated virus composition does not contain the adjuvants selected from aluminum salts, calcium phosphate, toll-like receptor (TLR) agonists, monophosphoryl lipid A (MLA), MLA derivatives, synthetic lipid A, lipid A mimetics or analogs, cytokines, saponins, muramyl dipeptide (MDP) derivatives, CpG oligos, lipopolysaccharide (LPS) of gram-negative bacteria, polyphosphazenes, emulsions (oil emulsions), chitosan, vitamin D, stearyl or octadecyl tyrosine, virosomes, cochleates, poly(lactide-co-glycolides) (PLG) microparticles, poloxamer particles, microparticles, liposomes, Complete Freund's Adjuvant (CFA), and Incomplete Freund's Adjuvant (IFA). In certain such embodiments, the liquid inactivated virus composition does not contain an adjuvant i.e. any adjuvant compound known to the skilled person.

In certain embodiments, the concentration of phosphate ions within the liquid inactivated virus composition is less than about 7 mM, or less than about 6 mM, or less than about 5 mM, or less than about 4 mM, or less than about 3 mM, or less than about 2 mM, or less than about 1 mM. A liquid comprising phosphate ions is e.g. obtainable by dissolving or dispersing Sodium Phosphate Dibasic (Na₂HPO₄) and/or Potassium Phosphate Monobasic (KH₂PO₄) in a liquid. When Sodium Phosphate Dibasic (Na₂HPO₄) and Potassium Phosphate Monobasic (KH₂PO₄) are dissolved in a particular ratio in an aqueous liquid this produces a phosphate buffer solution.

In certain embodiments, the concentration of the said at least one pharmaceutically acceptable buffer in the liquid inactivated virus composition is at least about 7 mM, or at least about 7.5 mM, or at least about 8 mM, or at least about 8.5 mM, or at least about 9 mM, or at least about 10 mM. In certain such embodiments, the concentration of the at least one pharmaceutically acceptable buffer in the liquid inactivated virus composition is from about 7 mM to about 200 mM, or from about 7.5 mM to about 200 mM, or from about 8 mM to about 200 mM, or from about 8.5 mM to about 200 mM, or from about 9 mM to about 100 mM, or from about 9 mM to about 60 mM, or from 9 mM to about 30 mM or from about 9 mM to about 11 mM or about 10 mM, or about 20 mM, or about 50 mM.

In certain embodiments, the liquid inactivated virus composition comprises only one pharmaceutical acceptable buffer. Additionally, in certain embodiments the liquid inactivated virus composition may comprise substantially only one pharmaceutically acceptable buffer and only residual amounts of additional buffer components, with a concentration of less than 2 mM, or less than 1.5 mM, or less than 1 mM or less than 0.9 mM, or less than 0.5 mM, or less than 0.2 mM.

In certain embodiments, the liquid inactivated virus composition comprises at least two different pharmaceutically acceptable buffers, wherein the molar ratio of the two most concentrated pharmaceutically acceptable buffers in the liquid inactivated virus composition is not between 1:2 to 2:1, or between 1:5 to 5:1, or between 8:1 to 1:8, or between 10:1 to 1:10.

In certain embodiments, the concentration of potassium ions in the liquid inactivated virus composition is less than about 4 mM, or less than about 3 mM, or less than about 2 mM, or less than about 1.5 mM, or less than about 0.5 mM, or less than about 0.1 mM, or about 0 mM (i.e. substantially free of potassium ions).

In certain embodiments, the liquid inactivated virus composition is substantially free or free of protamine sulphate.

In certain embodiments, the pH of the liquid inactivated virus composition is from about pH 6.0 to about pH 9.0 or from about pH 6.5 to about pH 8.0, or from about pH 6.8 to about pH 7.8, about pH 7.4 or about pH 7.6, as determined at room temperature.

### Buffer

In certain embodiments, the liquid inactivated virus composition according to the present invention comprises:
a) an inactivated whole Zika virus,
b) at least one pharmaceutically acceptable buffer with a concentration of at least about 6.5 mM, and
c) optionally a polyol,
   wherein the liquid inactivated virus composition does not contain an adjuvant selected from aluminum salts and
   said at least one pharmaceutically acceptable buffer comprises an amino group-containing molecule and does not comprise phosphate ions.

Buffers comprising amino group-containing molecules can be selected from the group of Histidine (His), Tris, ACES, CHES, CAPSO, TAPS, CAPS, Bis-Tris, TAPSO, TES, Tricine, and ADA. In certain preferred embodiments, the pharmaceutically acceptable buffer is Tris or Histidine (His) buffer, preferably Tris buffer.

### Polyol

In certain embodiments, the liquid inactivated virus composition further comprises at least one polyol.

In certain such embodiments, the liquid inactivated virus composition comprises from about 1% w/v to about 60% w/v of the polyol, or from about 6% w/v to about 50% w/v of the polyol, or from about 6% w/v to about 40% w/v of the polyol, or from about 6% w/v to about 35% w/v of the polyol, or from about 6% w/v to about 30% w/v of the polyol, or from about 6% w/v to about 25% w/v of the polyol, or from about 6% w/v to about 20% w/v of the polyol, or from about 6% w/v to about 15% w/v of the polyol, or from about 6% w/v to about 12% w/v of the polyol, or about 7% w/v of the polyol or about 10% w/v of the polyol.

In certain preferred embodiments, the liquid inactivated virus composition comprises a pharmaceutically acceptable buffer comprising an amino group-containing molecule, and from about 6% w/v to about 15% w/v of a polyol. In certain such embodiments, the liquid inactivated virus composition comprises Tris, and from about 6% w/v to about 15% w/v of a polyol.

In certain embodiments, the polyol is a sugar. In certain such embodiments, the sugar is a disaccharide. In certain such embodiments, the disaccharide is a non-reducing sugar. In certain such embodiments, the non-reducing sugar is sucrose.

In certain such embodiments, the liquid inactivated virus composition comprises from about 5% w/v to about 20% w/v sucrose, or from about 6% w/v to about 15% w/v sucrose. In certain such embodiments, the liquid inactivated virus composition comprises from about 6% w/v to about 8% w/v sucrose, such as about 7% w/v sucrose.

In a certain preferred aspect, the liquid inactivated virus composition comprises from about 8.5 mM to about 50 mM Tris and from about 6% to about 15% w/v sucrose, wherein the pH of the inactivated virus composition is from about pH 7.0 to about pH 8.0, when measured at room temperature.

In certain alternative embodiments, the polyol is glycerol. In certain such embodiments, the liquid inactivated virus composition comprises from about 1% v/v to about 60% v/v glycerol, or from about 7% v/v to about 15% v/v glycerol, or about 10% v/v of glycerol.

In a certain preferred aspect, the inactivated virus composition comprises from about 8.5 mM to about 50 mM Tris and from about 6% v/v to about 15% v/v glycerol, wherein the pH of the inactivated virus composition is from about pH 7.0 to about pH 8.0, when measured at room temperature.

### Sodium Chloride

In certain embodiments, the liquid inactivated virus composition further comprises sodium chloride. In certain such embodiments, the liquid inactivated virus composition comprises a concentration of sodium chloride of from about about 5 mM to about 500 mM sodium chloride, or from about 10 mM to about 200 mM.

In certain such embodiments, the liquid inactivated virus composition comprises a concentration of sodium chloride of from about 10 mM to about 40 mM, or from about 10 mM to about 30 mM, such as about 20 mM of sodium chloride. In a certain preferred embodiment, the liquid inactivated virus composition comprises from about 8.5 mM to about 80 mM Tris, from about 10 mM to about 30 mM sodium chloride, from about 6% to about 15% w/v sucrose, wherein the pH of the liquid inactivated virus composition is from about pH 7.0 to about pH 8.0, when measured at room temperature. In a certain preferred embodiment, the liquid inactivated virus composition comprises from about 8.5 mM to about 15 mM Tris, from about 10 mM to about 25 mM sodium chloride, from about 6% to about 10% w/v sucrose, wherein the pH of the liquid inactivated virus composition is from about pH 7.0 to about pH 8.0, when measured at room temperature.

In certain alternative embodiments, the liquid inactivated virus composition comprises a concentration of sodium chloride of from about 100 mM to about 200 mM, or from about 140 mM to about 160 mM, such as about 150 mM. In certain such embodiments, the liquid inactivated virus composition comprises from about 8.5 mM to about 80 mM Tris and from about 140 mM to about 160 mM NaCl, wherein the pH of the liquid inactivated virus composition is from about pH 7.0 to about pH 8.0, when measured at room temperature. In certain such embodiments, the liquid inactivated virus composition comprises from about 8.5 mM to about 15 mM Tris and from about 140 mM to about 160 mM NaCl, wherein the pH of the liquid inactivated virus composition is from about pH 7.0 to about pH 8.0, when measured at room temperature.

In certain embodiments, the ionic strength of the liquid inactivated virus composition is below about 80 mM, or below about 70 mM, or below about 60 mM, or below about 50 mM, or below about 40 mM, or below about 30 mM. The term ionic strength is defined by the following equation:

$$I = \frac{1}{2} {\sum }_{i = 1}^{n} {C}_{i} {Z}_{i}^{2}$$

Where, Cᵢ is the molar concentration of the ion I, Zᵢ is the charge number of that ion, and the sum is taken over all ions in the solution.

### Zika Virus

The present invention relates to an inactivated virus composition comprising an inactivated whole Zika virus. In certain aspects of the present invention, the inactivated whole Zika virus may refer to a purified inactivated whole Zika virus isolated by plaque purification from a population of Zika viruses. The invention relates to any type of inactivated whole Zika virus. Below a specific Zika virus is described as an example.

Zika virus (ZIKV) is a mosquito-borne flavivirus first isolated from a sentinel rhesus monkey in the Zika Forest in Uganda in 1947. Since that time, isolations have been made from humans in both Africa and Asia, and more recently, the Americas. ZIKV is found in two (possibly three) lineages: an African lineage (possibly separate East and West African lineages) and an Asian lineage. Accordingly, examples of suitable Zika viruses of the present disclosure include, without limitation, viruses from the African and/or Asian lineages. In some embodiments, the Zika virus is an African lineage virus. In some embodiments, the Zika virus is an Asian lineage virus. Additionally, multiple strains within the African and Asian lineages of Zika virus have been previously identified. Any one or more suitable strains of Zika virus known in the art may be used in the present disclosure, including, for examples, strains Mr 766, ArD 41519, IbH 30656, P6-740, EC Yap, FSS13025, ArD 7117, ArD 9957, ArD 30101, ArD 30156, ArD 30332, HD 78788, ArD 127707, ArD 127710, ArD 127984, ArD 127988, ArD 127994, ArD 128000, ArD 132912, 132915, ArD 141170, ArD 142623, ArD 149917, ArD 149810, ArD 149938, ArD 157995, ArD 158084, ArD 165522, ArD 165531, ArA 1465, ArA 27101, ArA 27290, ArA 27106, ArA 27096, ArA 27407, ArA 27433, ArA 506/96, ArA 975-99, Ara 982-99, ArA 986-99, ArA 2718, ArB 1362, Nigeria68, Malaysia66, Kedougou84, Suriname, MR1429, PRVABC59, ECMN2007, DakAr41524, H/PF/2013, R103451, 103344, 8375, JMB-185, ZIKV/H, sapiens/Brazil/Natal/2015, SPH2015, ZIKV/Hu/Chiba/S36/2016, and/or Cuba2017. In some embodiments, strain PRVABC59 is used in the present disclosure.

In some embodiments, an example of a Zika virus genome sequence is set forth below as SEQ ID NO: 2:

```
        1 gttgttgatc tgtgtgaatc agactgcgac agttcgagtt tgaagcgaaa gctagcaaca
      61 gtatcaacaa gttttatttt ggatttggaa acgagaattt ctgatcataa aaaacccaaa
    121 aaagaaatcc ggaggattcc ggattgtcaa tatgctaaaa cgcggagtag cccgtgtgag
    181 cccctttagg ggcttgaaga ggctgccagc cggacttctg ctgggtcatg ggcccatcag
    241 gatggtcttg gcgattctag ccttttagag attcacggca atcaagccat cactgggtct
    301 catcaataga tggggttcag tggggaaaaa agaggctatg gaaacaataa agaagttcaa
    361 gaaagatctg gctgccatgc tgagootaat caatgctagg aaggagaaga agagacgagg
    421 cgcagatact agtgtcgaaa ttgttggcct cctactaacc acagctatgg cagcggaggt
    481 cactagacgt gagagtgcat actatatgta caggacaga aacgatgctg gggaggccat
    541 attttttcca accacattgg ggatgaataa gtgttatata cagatcatgg atcttggaca
    601 catgtgtgat gccaccatga gctatgaatg ccctatgctg gatgaggggg tggaaccaga
    661 tgacgtcgat tgttggtgca acacgacgtc aacttgggtt gtgtacggaa cctgccatca
    721 caaaaaaggt gaagcacgga gatctagaag agctgtgacg ctcccctccc attccaccag
    781 gaagctgcaa acgcggtcgc aaacctggtt ggaatcaaga gaatacacaa agcacttgat
    841 tagagtcgaa aattggatat tcaggaaccc tggcttcgcg ttagcagcag ctgccatcgc
    901 ttggcttttg ggaagctcaa cgagccaaaa agtcatatac ttggtcatga tactgctgat
    961 tgccccgaca tacagcatca ggtacatagg agtcagcaat aggaactttg tggaaggtat
  1021 atcaggtggg acttgagttg atgttgtctt agaacatgga gattatgtca ccgtaatggc
  1081 acaggacaaa ccgactgtcg acatagagct ggttacaaca acagtcagca acatggcgga
  1141 ggtaagatcc tactgctatg aggcatcaat atcagacatg gcttctgaca gccgctgccc
  1201 aacacaaggt gaagcctacc ttgacaagca atcagacact caatatgtct gcaaaagaac
  1261 gttagtggac agaggctggg gaaatggatg tggacttttt ggcaaaggga gcctggtgac
  1321 atgcgctaag tttgcatgct ccaagaaaat gaccgggaag agcatccagc cagagaatct
  1381 ggagtaccgg ataatgctgt cagttcatgg ctcccagcac agtgggatga tcgttaatga
  1441 cacaggacat gaaactgatg agaatagagc gaaagttgag ataacgccca attcaccgag
  1501 agccgaagcc accctgggga gttttggaag cctaggactt gattgtgaac cgaggacagg
  1561 ccttgacttt tcagatttgt attacttgac tatgaataac aagcactggt tggttcacaa
  1621 ggagtggttc cacgacattc cattaccttg gcacgctggg gcagacaccg gaactccaca
  1681 ctggaacaac aaagaagcac tggtagagtt caaggacgca catgccaaaa ggcaoactgt
  1741 catggttcta gggagtcaag aaggagcagt tcacacggcc cttgctggag ctctgaaggc
  1801 tgagatggat ggtgcaaagg gaaggctgtc ctctggccac ttgaaatgtc gcctgaaaat
  1861 ggataaactt agattgaagg gcgtgtcata ctccttgtgt actgcagcgt tcacattcac
  1921 caagatcccg gctgaaacac tgcacgggac agtcacagtg gaggtacagt acgcagggac
  1981 agatggacct tgcaaagttc caactcagat ggcggtagac atgcaaactc tgaccccagt
  2041 tgggaggttg ataaccgcta accccgtaat cactgaaagc actgagaact ctaagatgat
  2101 gctggaactt gatccaccat ttggggactc ttacattgtc ataggagtcg gggagaagaa
  2161 gatcacccac cactggcaca ggagtggcag caccattgga aaagcatttg aagccactgt
  2221 gagaggtgcc aagagaatgg cagtcttggg agacacagcc tgggactttg gatcagttgg
  2281 aggcgctctc aactcattgg acaagggcat ccatcaaatt tttagagcag ctttcaaatc
  2341 attgtttgga ggaatgtcct ggttctcaca aattctcatt ggaacgttgc tgatgtggtt
  2401 gggtctaaac acaaagaata gatctatttc ccttatatgc ttggccttaa ggaaagtgtt
  2461 gatcttctta tccacagccg tctctgctga tgtgaggtgc tcgatggact tctcaaagaa
  2521 ggagacgaga tgcggtacag gggtgttcgt ctataacgac gttgaagcct ggagggacag
  2581 gtacaagtac catcctgact ccccccgtag attggcagca gcagtcaagc aagcctggga
  2641 agatggtatc tgcaggatct cctctgtttc aagaatggaa aacatcatat agaaatcagt
  2701 agaagggaag ctcaacgcaa tcctggaaaa aaatggagtt caactaacag tcgttgtggg
  2761 atctgtaaaa aaccccatgt ggagaggtcc acagagattg cccgtgcctg tgaacaagct
  2821 gccccacggc tggaaggctt gggggaaatc gtatttcgtc agagcagcaa agacaaataa
  2881 cagctttgtc gtggatggtg acacactgaa ggaatgccca ctcaaacata gagcatggaa
  2941 cagctttctt atggaggatc atgggttcag aatatttcac actaatgtct ggctcaaggt
  3001 tagaaaaaat tattcattag agtgtgatcc agccgttatt ggaacagctg ttaaaggaaa
  3061 ggaggctgta cacagtgatc taggctactg gattgagagt gagaagaatg acacatggag
  3121 gctgaagagg gcccatctga tcgagatgaa aacatgtgaa tggccaaagt cccacacatt
  3181 atggacagat gaaatagaaa aaagtgatct gatcataccc aagtctttag ctgggccact
  3241 cagccatcac aataccagag aggactacag aacccaaatg aaaaagccat ggcacagtga
  3301 agaacttgaa attcggtttg aggaatgccc aggcactaag gtccacgtgg aggaaacatg
  3361 tggaacaaga ggaccatctc tgagatcaac cactgcaagc ggaagggtga tcgaggaatg
  3421 gtgctgcagg gagtgcacaa tgcccccact gtcgttccgg gctaaagatg gctgttggta
  3481 tgaaatggag ataagaccca ggaaaaaacc aaaaagcaac ttagtaagat caatgatgac
  3541 tgcaagatca actaatcaca tgaaccactt ctcccttgga gtgcttgtga tcctgctcat
  3601 ggtgcaggaa gggctgaaga agagaatgac cacaaagatc atcataagca catcaatggc
  3661 agtgctggta gctatgatcc tgggaggatt ttcaatgagt gacctggcta agcttgcaat
  3721 tttgatgggt gccaccttcg cggaaatgaa cactgaagga aatataactc atctgacgct
  3781 aatagcagca ttcaaagtca gaccagcgtt gctggtatct ttcatcttca gagctaattg
  3841 gacaccccgt gaaagcatgc tgctggcctt ggcctcgtgt cttttgcaaa ctgcgatctc
  3901 cgccttggaa ggcgacctga tggttctcat caatggtttt gctttggcct ggttggcaat
  3961 acgaacgatg gttgttccac gcactgataa catcacctta gcaatcctga ctgctctgac
  4021 accactggcc cggggcacac tgcttgtggc gtggagagca ggccttgcta cttgcggggg
  4081 gtttatgctc ctctctctga agggaaaagg cagtgtgaag aagaacttac catttgtcat
  4141 ggccctggga ctaaccgctg tgaggctggt cgaccccatc aacgtggtgg gactgctgtt
  4201 actcacaagg agtgggaagc ggagctggcc ccctagcaaa gtactcacag ctattgacct
  4261 aatatgcgca ttggctggag ggttcgccaa ggcaaatata gagatggctg ggcccatggc
  4321 cgcagtcggt ctgctaatta tcagttacat ggtctcagga aagagtatgg acatgtacat
  4381 tgaaagagca ggtgacatca catgggaaaa agatgcggaa gtcactggaa acagtccccg
  4441 gctcgatgtg gcgctagatg agagtggtga tttctccctg gtggaggatg acggtccccc
  4501 catgagaaag atcatactca agatggtcct aataaccatc tatggcataa acccaatagc
  4561 catacccttt gcagctggag cgtagtacgt atacgtgaaa actagaaaaa ggaatggtgc
  4621 tctatggaat gtacctgctc ccaaggaagt aaaaaagggg gagaccacag atggagtgta
  4681 cagagtaatg actcgtagac tgctaggttc aacacaagtt ggagtgggag ttatgcaaga
  4741 gggggtcttt cacactatgt ggcacgtcac aaaaggatcc gcgctgagaa gcggtgaagg
  4801 gagacttgat ccatactggg gagatgtcaa gcaggatctg gtgtcatact gtggtccatg
  4861 gaagctagat accacctggg atgggcacag cgaggtgcag ctcttagccg tgccccccgg
  4921 agagagagcg aggaacatcc agactctgcc cgaaatattt aagacaaagg atgaggacat
  4981 tggagcggtt gcgctggatt acccagcagg aacttcagga tctccaatcc tagacaagtg
  5041 tgggagagtg ataggacttt atggcaatgg ggtcgtgatc aaaaacggga gttatgttag
  5101 tgccatcacc caagggagga gggaggaaga gactcctgtt gagtgcttcg agccctcgat
  5161 gctgaagaag aagcagctaa ctgtcttaga cttgcatcct ggagctggga aaaccaggag
  5221 agttcttcct gaaatagtcc gtgaagccat aaaaacaaga ctccgtactg tgatcttagc
  5281 tccaaccagg gttgtcgctg ctgaaatgga ggaggccctt agagggcttc cagtgcgtta
  5341 tatgacaaca gcagtcaatg tcacccactc tggaacagaa atcgtcgact taatgtgcca
  5401 tgccaccttc acttcacgtc tactacagcc aatcagaatc cccaactata atctgtatat
  5461 tatggatgag gcccacttca cagatccctc aagtataaca gcaagaggat acatttcaac
  5521 aagggttgag atgggcgagg cggctgccat cttcatgacc gccacgccac caggaacccg
  5581 tgacgcattt ccggactcca actcaccaat tatggacacc gaagtggaag tcccagagag
  5641 agcctggagc tcaggctttg attgggtgac ggatcattct ggaaaaacag tttggtttgt
  5701 tccaagcgta aggaacggca atgagatcgc aacttgtctg acaaagacta gaaaacgggt
  5761 catacagctc agcagaaaga cttttgagac agagttccag aaaacaaaac atcaagagtg
  5821 ggactttgtc gtgacaactg acatttcaga gatgggcgcc aactttaaag ctgaccgtgt
  5881 catagattcc aggagatgcc taaagccggt catacttgat ggcgagagag tcattctggc
  5941 tggacccatg cctgtcacac atgccagcgc tgcccagagg agggggcgca taggcaggaa
  6001 tcccaacaaa cctggagatg agtatctgta tggaggtggg tgcgcagaga ctgacgaaga
  6061 ccatgcacac tggcttgaag caagaatgct ccttgacaat atttacctcc aagatggcct
  6121 catagcctcg ctctatcgac ctgaggccga caaagtagca gccattgagg gagagttcaa
  6181 gcttaggacg gagcaaagga agacctttgt ggaactcatg aaaagaggag atcttcctgt
  6241 ttggctggcc tatcaggttg catctgccgg aataacctac acagatagaa gatggtgctt
  6301 tgatggcacg accaacaaca ccataatgga agacagtgtg ccggcagagg tgtggaccag
  6361 acacggagag aaaagagtgc tcaaaccgag gtggatggac gccagagttt gttcagatca
  6421 tgcggcccta aagtcattca aggagtttac cgctggaaaa agaggagcgg cttttggagt
  6481 gatggaagcc ctgggaacac tgccaggaca catgacagag agattccagg aagccattga
  6541 caacctcgct gtgctcatgc gggcagagac tggaagcagg ccttacaaag ccgcggcggc
  6601 ccaattgccg gagaccctag agaccataat gcttttgggg ttgctgggaa cagtctcgct
  6661 gggaatcttc ttcgtcttga tgaggaacaa gggcataggg aagatgggct ttggaatggt
  6721 gactcttggg gccagcgcat ggctcatgtg actctcggaa attgagccag ccagaattgc
  6781 atgtgtcctc attgttgtgt tcctattgct ggtggtgctc atacctgagc cagaaaagca
  6841 aagatctccc caggacaacc aaatggcaat catcatcatg gtagcagtag gtcttctggg
  6901 cttgattacc gccaatgaac tcggatggtt ggagagaaca aagagtgacc taagccatct
  6961 aatgggaagg agagaggagg gggcaaccat aggattctca atggacattg acctgcggcc
  7021 agcctcagct tgggccatct atgctgcctt gacaactttc attaccccag ccgtccaaca
  7081 tgcagtaacc acctcataca acaactactc cttaatgacg atggccacgc aagctggagt
  7141 attatttggc atgggcaaag ggatgccatt ctacgcatgg gactttagag tcccactact
  7201 aatgatagct tgctactcac aattaacacc cctgacccta atagtggcca tcattttgct
  7261 cgtggcgcac tacatgtact tgatcccagg gctgcaggca gcagctgcgc gtgctgccca
  7321 gaagagaacg gcagctggca tcatgaagaa ccctgttgtg gatggaatag tggtgactga
  7381 cattgacaca atgacaattg acccccaagt ggagaaaaag ataggacagg tgctactcat
  7441 agcagtagcc gtctccagcg ccatactgtc gcggaccgcc tgggggtggg gggaggctgg
  7501 ggctctgatc acagccgcaa cttccacttt gtgggaaggc tctccgaaca agtactggaa
  7561 ctcctctaca gccacttcac tgtgtaacat ttttagggga agttacttgg ctggagcttc
  7621 tctaatctac acagtaacaa gaaacgctga cttgatcaaa agacatggag gtggaacagg
  7681 agagaccctg ggagagaaat agaaggcccg cttgaaccag atgtcgaccc tggagttcta
  7741 ctcctacaaa aagtcaggca tcaccgaggt gtgcagagaa gaggcccgcc gcgccctcaa
  7801 ggacggtgtg gcaacgggag gccatgctgt gtcccgagga agtgcaaagc tgagatggtt
  7861 ggtggagcgg ggatacctgc agccctatgg aaaggtcatt gatcttggat gtggcagagg
  7921 gggctgaagt tactacgtcg ccaccatcca caaagttcaa gaagtgaaag gatacacaaa
  7981 aggaggccct ggtcatgaag aacccgtgtt ggtgcaaagc tatgggtgga acatagtccg
  8041 tcttaagagt ggggtggacg tctttcatat ggcggctgag ccgtgtgaca cgttgctgtg
  8101 tgacataggt gagtcatcat ctagtcctga agtggaagaa gcacggacgc tcagagtcct
  8161 ctccatggtg agggattgac ttgaaaaaag accaggagcc ttttgtataa aagtgttgtg
  8221 cccatacacc agcactatga tggaaaccct ggagcgactg cagcgtaggt atgggggagg
  8281 actggtcaga gtgccactct cccgcaactc tacacatgag atgtactggg tctctggagc
  8341 gaaaagcaac accataaaaa gtgtgtccac cacgagccag ctcctcttgg ggcgcatgga
  8401 caggcctagg aggccagtga aatatgagga agatatgaat ctcagctcta gcacgcgagc
  8461 tgtggtaagc tgcgctgaag ctcccaacat gaagatcatt ggtaaccgca ttgaaaggat
  8521 ccgcagtgag cacgcggaaa cgtggttctt tgacgagaac cacccatata ggacatgggc
  8581 ttaccatgga agctatgagg cccccacaca agggtcagcg tcctctctaa taaacggggt
  8641 tgtcagactc ctgtcaaaac cctaggatgt ggtgactgga atcacaggaa taaccataac
  8701 cgacaccaca ccgtatggtc agcaaagagt tttcaaggaa aaagtggaca ctagggtgcc
  8761 agacccccaa gaaggcactc gtcaggttat gagcatggtc tcttcctggt tgtggaaaga
  8821 gctaggcaaa cacaatcggc cacgagtctg caccaaagaa gagttcatca acaaggttcg
  8881 tagcaatgca gcattagggg caatatttga agaggaaaaa gagtggaaga ctgcagtgga
  8941 aactgtgaac gatccaaggt tctaggctct agtgaacaag gaaagagagc accacctgag
  9001 aggagagtgc cagagctgtg tgtacaacat gatgggaaaa agagaaaaga aacaagggga
  9061 atttggaaag gccaagggca gccgcgccat ctggtatatg tggctagggg ctagatttct
  9121 agagttcgaa gcccttggat tcttgaacga ggatcactcg atggggagag agaactcagg
  9181 aggtggtgtt gaagggctgg gattacaaag actcggatat gtcctagaag agatgagtcg
  9241 tataccagga ggaaggatgt atgcagatga cactgctggc tgggacaccc gcattagcag
  9301 atttgatctg gagaatgaag ctctaatcac caaccaaatg gagaaagggc acagggcctt
  9361 ggcattggcc ataatcaagt acacatacca aaacaaagtg gtaaaggtcc ttagaccagc
  9421 tgaaaaaggg aaaacagtta tggacattat ttcgagacaa gaccaaaggg ggaacggaca
  9481 agttgtcact tacgctctta acacatttac caacctagtg gtgcaactca ttcggaatat
  9541 ggaggctgag gaagttctag agatgcaaga cttgtggctg ctgcggaggt cagagaaagt
  9601 gaccaactga ttgcagagca acggatggga taggctcaaa cgaatggcag tcagtggaga
  9661 tgattgcgtt gtgaagccaa ttgatgatag gtttgcacat gccctcaggt tcttgaatga
  9721 tatgggaaaa gttaggaagg acacacaaga gtggaaacce tcaactggat gggacaactg
  9781 ggaagaagtt ccgttttgct cccaccactt caacaagctc catctcaagg acgggaggtc
  9841 cattgtggtt ccctgccgcc accaagatga actgattggc cgggcccacg tctctccagg
  9901 ggcgggatgg agcatccggg agactgctta cctagcaaaa tcatatacgc aaatgtggca
  9961 gctcctttat ttccacagaa gggacctccg actgatggcc aatgccattt gttcatctat
10021 gccagttgac tgggttccaa ctgggagaac tacctggtca atccatggaa agggagaatg
10081 gatgaccact gaagacatgc ttgtggtgtg gaacagagtg tggattgagg agaacgacca
10141 catggaagac aagaccccag ttacgaaatg gacagacatt ccctatttga gaaaaaggga
10201 agacttgtag tgtggatctc tcatagggca cagaccgcac accacctggg ctgagaacat
10261 taaaaacaca gtcaacatgg tgcgcaggat cataggtgat gaagaaaagt acatggacta
10321 cctatccacc caagttcgct acttgggtga agaagggtct acacctggag tgctgtaagc
10381 accaatctta atgttgtcag gcctactagt cagccacaac ttgaggaaaa ctgtgcagcc
10441 tgtgaccccc ccaggagaag ctgggaaacc aagcctatag tcaggccgag aacgccatgg
10501 cacggaagaa gccatgctgc ctgtgagccc ctcagaggac actgagtcaa aaaaccccac
10561 gcgcttggag gcgcaggatg ggaaaagaag gtggcgacct tccccaccct tcaatctggg
10621 acctgaactg gagatcagct gtgtatctcc agaagaggga ctagtggtta gagga
```

In some embodiments, the Zika virus may comprise the genome sequence of GenBank Accession number KU501215.1. In some embodiments, the Zika virus is from strain PRVABC59. In some embodiments the genome sequence of GenBank Accession number KU501215.1 comprises the sequence of SEQ ID NO: 2. In some embodiments, the Zika virus may comprise a genomic sequence that has at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the sequence of SEQ ID NO: 2.

In some embodiments, the Zika virus may comprise at least one polypeptide encoded by the sequence of SEQ ID NO: 2. In some embodiments, the Zika virus may comprise at least one polypeptide having an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with an amino acid sequence encoded by the sequence of SEQ ID NO: 2.

Accordingly, in some embodiments, inactivated Zika viruses of the present disclosure may be used in any of the inactivated virus compositions disclosed herein. For example, inactivated Zika viruses of the present disclosure may be used to provide one or more antigens useful for treating or preventing Zika virus infection in a subject in need thereof and/or for inducing an immune response, such as a protective immune response, against Zika virus in a subject in need thereof.

The Zika virus used in the present disclosure may be obtained from one or more cells in cell culture (e.g., via plaque purification). Any suitable cells known in the art for producing Zika virus may be used, including, for example, insect cells (e.g., mosquito cells such as CCL-125 cells, Aag-2 cells, RML-12 cells, C6/36 cells, C7-10 cells, AP-61 cells, A.t. GRIP-1 cells, A.t. GRIP-2 cells, A.t. GRIP-3 cells, UM-AVE1 cells, Mos.55 cells, Sua1B cells, 4a-3B cells, Mos.42 cells, MSQ43 cells, LSB-AA695BB cells, NIID-CTR cells, TRA-171, cells, and additional cells or cell lines from mosquito species such as Aedes aegypti, Aedes albopictus, Aedes pseudoscutellaris, Aedes triseriatus, Aedes vexans, Anopheles gambiae, Anopheles stephensi, Anopheles albimus, Culex quinquefasciatus, Culex theileri, Culex tritaeniorhynchus, Culex bitaeniorhynchus, and/or Toxorhynchites amboinensis), and mammalian cells (e.g., VERO cells (from monkey kidneys), LLC-MK2 cells (from monkey kidneys), MDBK cells, MDCK cells, ATCC CCL34 MDCK (NBL2) cells, MDCK 33016 (deposit number DSM ACC 2219 as described in WO97/37001) cells, BHK21-F cells, HKCC cells, or Chinese hamster ovary cells (CHO cells). In some embodiments, the Zika virus (e.g., a Zika virus clonal isolate) is produced from a non-human cell. In some embodiments, the Zika virus (e.g., a Zika virus clonal isolate) is produced from an insect cell. In some embodiments, the Zika virus (e.g., a Zika virus clonal isolate) is produced from a mosquito cell. In some embodiments, the Zika virus (e.g., a Zika virus clonal isolate) is produced from a mammalian cell. In some embodiments, the Zika virus (e.g., a Zika virus clonal isolate) is produced from a VERO cell.

Zika viruses possess a positive sense, single-stranded RNA genome encoding both structural and nonstructural polypeptides. The genome also contains non-coding sequences at both the 5′- and 3′-terminal regions that play a role in virus replication. Structural polypeptides encoded by these viruses include, without limitation, capsid (C), precursor membrane (prM), and envelope (E). Non-structural (NS) polypeptides encoded by these viruses include, without limitation, NS1, NS2A, NS2B, NS3, NS4A, NS4B, and NS5.

In certain embodiments, the Zika virus includes a mutation in Zika virus Non-structural protein 1 (NS1). In some embodiments, the Zika virus contains a Trp98Gly mutation at position 98 of SEQ ID NO: 1, or at a position corresponding to position 98 of SEQ ID NO: 1.

In some embodiments, the mutation is within the NS1 polypeptide. The amino acid sequence of a wild-type, NS1 polypeptide from an exemplary Zika virus strain is set forth as:

(SEQ ID NO: 1)
DVGCSVDFSKKETRCGTGVFVYNDVEAWRDRYKYHPDSPRRLAAAVKQAW
EDGICGISSVSRMENIMWRSVEGELNAILEENGVQLTVVVGSVKNPMWRG
PQRLPVPVNELPHGWKAWGKSYFVRAAKTNNSFVVDGDTLKECPLKHRAW
NSFLVEDHGFGVFHTSVWLKVREDYSLECDPAVIGTAVKGKEAVHSDLGY
WIESEKNDTWRLKRAHLIEMKTCEWPKSHTLWTDGIEESDLIIPKSLAGP
LSHHNTREGYRTQMKGPWHSEELEIRFEECPGTKVHVEETCGTRGPSLRS
TTASGRVIEEWCCRECTMPPLSFRAKDGCWYGMEIRPRKEPESNLVRSMV
T.

In some embodiments, the amino acid sequence of the NS1 polypeptide has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with the sequence of SEQ ID NO: 1. In some embodiments, the amino acid sequence of the NS1 polypeptide may be from the amino acid sequence encoded by the sequence of GenBank Accession number KU501215.1 (SEQ ID NO: 2). In some embodiments, the amino acid sequence of the NS1 polypeptide may be amino acid positions 795 to 1145 of the amino acid sequence encoded by the sequence of GenBank Accession number KU501215.1. In some embodiments, the amino acid sequence of the NS1 polypeptide may be from Zika virus strain PRVABC59.

“Sequence Identity”, “% sequence identity”, “% identity”, “% identical” or “sequence alignment” means a comparison of a first amino acid sequence to a second amino acid sequence, or a comparison of a first nucleic acid sequence to a second nucleic acid sequence and is calculated as a percentage based on the comparison. The result of this calculation can be described as “percent identical” or “percent ID.”

Generally, a sequence alignment can be used to calculate the sequence identity by one of two different approaches. In the first approach, both mismatches at a single position and gaps at a single position are counted as non-identical positions in final sequence identity calculation. In the second approach, mismatches at a single position are counted as non-identical positions in final sequence identity calculation; however, gaps at a single position are not counted (ignored) as non-identical positions in final sequence identity calculation. In other words, in the second approach gaps are ignored in final sequence identity calculation. The difference between these two approaches, i.e. counting gaps as non-identical positions vs ignoring gaps, at a single position can lead to variability in the sequence identity value between two sequences.

In some embodiments, a sequence identity is determined by a program, which produces an alignment, and calculates identity counting both mismatches at a single position and gaps at a single position as non-identical positions in final sequence identity calculation. For example program Needle (EMBOS), which has implemented the algorithm of Needleman and Wunsch (Needleman and Wunsch, 1970, J. Mol. Biol. 48: 443-453), and which calculates sequence identity per default settings by first producing an alignment between a first sequence and a second sequence, then counting the number of identical positions over the length of the alignment, then dividing the number of identical residues by the length of an alignment, then multiplying this number by 100 to generate the % sequence identity [% sequence identity=(# of Identical residues/length of alignment)×100)].

A sequence identity can be calculated from a pairwise alignment showing both sequences over the full length, so showing the first sequence and the second sequence in their full length (“Global sequence identity”). For example, program Needle (EMBOSS) produces such alignments; % sequence identity=(# of identical residues/length of alignment)×100)].

A sequence identity can be calculated from a pairwise alignment showing only a local region of the first sequence or the second sequence (“Local Identity”). For example, program Blast (NCBI) produces such alignments; % sequence identity=(# of Identical residues/length of alignment)×100)].

The sequence alignment is preferably generated by using the algorithm of Needleman and Wunsch (J. Mol. Biol. (1979) 48, p. 443-453). Preferably, the program “NEEDLE” (The European Molecular Biology Open Software Suite (EMBOSS)) is used with the programs default parameter (gap open=10.0, gap extend=0.5 and matrix=EBLOSUM62 for proteins and matrix=EDNAFULL for nucleotides). Then, a sequence identity can be calculated from the alignment showing both sequences over the full length, so showing the first sequence and the second sequence in their full length (“Global sequence identity”). For example: % sequence identity=(# of identical residues/length of alignment)×100)].

In some embodiments, a mutation occurs at one or more amino acid positions within the NS1 polypeptide. In some embodiments, the mutation occurs at position 98 of SEQ ID NO: 1, or at a position corresponding to position 98 of SEQ ID NO: 1 when aligned to SEQ ID NO: 1 using a pairwise alignment algorithm. In some embodiments, the mutation at position 98 is a tryptophan to glycine substitution.

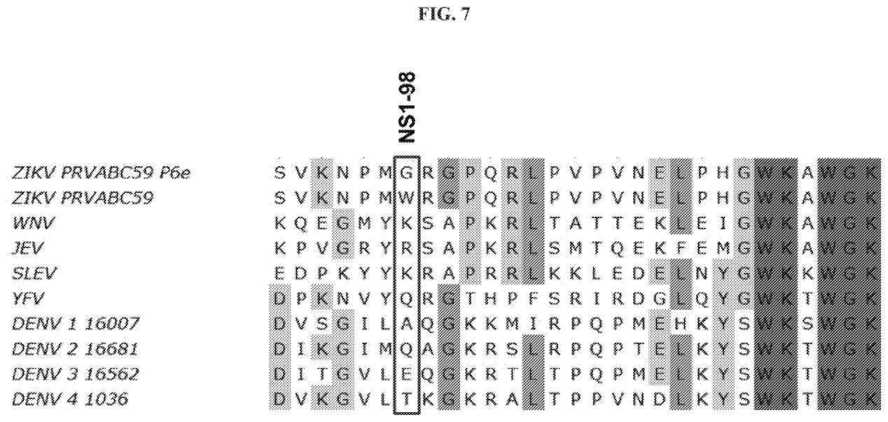

In some embodiments, the Zika virus comprises a mutation at position 98 of SEQ ID NO: 1, or at a position corresponding to position 98 of SEQ ID NO: 1. A position corresponding to position 98 of SEQ ID NO: 1 can be determined by aligning the amino acid sequence of an NS1 protein to SEQ ID NO: 1 using a pairwise alignment algorithm. Amino acid residues in viruses other than Zika virus, which correspond to the tryptophan residue at position 98 of SEQ ID NO: 1 are shown in FIG. 7 of the present application where these residues are boxed. In some embodiments, the mutation at position 98 is a tryptophan to glycine substitution. In some embodiments, the mutation at position 98 is a tryptophan to glycine substitution at position 98 of SEQ ID NO: 1. In some embodiments, the mutation at position 98 is a tryptophan to glycine substitution at a position corresponding to position 98 of SEQ ID NO: 1 when aligned to SEQ ID NO: 1 using a pairwise alignment algorithm.

In some embodiments, the Zika virus contains a mutation within the NS1 protein, and at least one mutation within one or more of the C, prM, E, NS1, NS2A, NS2B, NS3, NS4A, NS4B, and NS5 viral proteins. In some embodiments, the Zika virus contains one or more mutations within the NS1 protein, and does not contain at least one mutation within one or more of the C, prM, E, NS1, NS2A, NS2B, NS3, NS4A, NS4B, and NS5 viral proteins. In some embodiments, the Zika virus contains a mutation within the NS1 protein and does not contain at least one mutation within the envelope protein E. In some embodiments, whole, inactivated virus contains at least one mutation in Zika virus Non-structural protein 1 (NS1), and does not include a mutation in Zika virus envelope protein E (Env). In some embodiments, the Zika virus contains a mutation at position 98 of SEQ ID NO: 1, or at a position corresponding to position 98 of SEQ ID NO: 1 and does not contain any mutation within the envelope protein E. In some embodiments, whole, inactivated Zika virus contains a mutation at position 98 of SEQ ID NO: 1, or at a position corresponding to position 98 of SEQ ID NO: 1 and/or does not include a mutation in Zika virus envelope protein E (Env). In some embodiments, whole, inactivated virus contains at least one mutation in Zika virus Non-structural protein 1 (NS1) and the sequence encoding the envelope protein is the same as the corresponding sequence in SEQ ID No. 2. In some embodiments, the Zika virus contains a mutation at position 98 of SEQ ID NO: 1, or at a position corresponding to position 98 of SEQ ID NO: 1 and the sequence encoding the envelope protein is the same as the corresponding sequence in SEQ ID NO. 2. In some embodiments, whole, inactivated Zika virus contains a mutation at position 98 of SEQ ID NO: 1, or at a position corresponding to position 98 of SEQ ID NO: 1 and the sequence encoding the envelope protein is the same as the corresponding sequence in SEQ ID NO: 2. In some embodiments, whole, inactivated Zika virus contains a tryptophan to glycine substitution at position 98 of SEQ ID NO: 1, or at a position corresponding to position 98 of SEQ ID NO: 1 and the sequence encoding the envelope protein is the same as the corresponding sequence in SEQ ID NO: 2.

In some embodiments, the Zika virus contains at least one mutation that enhances genetic stability as compared to a Zika virus lacking the at least one mutation. In some embodiments, the Zika virus contains at least one mutation that enhances viral replication as compared to a Zika virus lacking the at least one mutation. In some embodiments, the Zika virus contains at least one mutation that reduces or otherwise inhibits the occurrence of undesirable mutations, such as within the envelope protein E (Env) of the Zika virus.

In the above embodiments of the present disclosure, an exemplary pairwise alignment algorithm is the Needleman-Wunsch global alignment algorithm, using default parameters (e.g. with Gap opening penalty=10.0, and with Gap extension penalty=0.5, using the EBLOSUM62 scoring matrix). This algorithm is conveniently implemented in the needle tool in the EMBOSS package.

In some embodiments, the inactivated Zika virus may be used in inactivated virus compositions. For example, the inactivated Zika virus may be useful for treating or preventing Zika virus infection in a subject in need thereof and/or inducing an immune response, such as a protective immune response, against Zika virus in a subject in need thereof.

### Production of Inactivated Virus Compositions

Other aspects of the present disclosure relate to Zika virus inactivated virus compositions containing a purified inactivated whole virus, such as a Zika virus with a mutation which is a tryptophan to glycine substitution at position 98 of SEQ ID NO: 1 or at a position corresponding to position 98 of SEQ ID NO: 1 as described herein. In some embodiments, the inactivated virus composition comprises a purified inactivated whole Zika virus comprising a Trp98Gly mutation at position 98 of SEQ ID NO: 1, or at a position corresponding to position 98 of SEQ ID NO: 1, wherein the Zika virus is derived from strain PRVABC59. In some embodiments, the inactivated virus composition comprises a purified inactivated whole Zika virus comprising a Trp98Gly mutation at position 98 of SEQ ID NO: 1, or at a position corresponding to position 98 of SEQ ID NO: 1, wherein the Zika virus is derived from strain PRVABC59 comprising the genomic sequence according to SEQ ID NO: 2. In one embodiment, the inactivated virus compositions contain a plaque purified clonal Zika virus isolate.

Production of inactivated virus compositions of the present disclosure includes growth of Zika virus. Growth in cell culture is a method for preparing inactivated virus compositions of the present disclosure. Cells for viral growth may be cultured in suspension or in adherent conditions.

Cell lines suitable for growth of the at least one virus of the present disclosure include, but are not limited to: insect cells (e.g., mosquito cells as described herein, VERO cells (from monkey kidneys), horse, cow (e.g. MDBK cells), sheep, dog (e.g. MDCK cells from dog kidneys, ATCC CCL34 MDCK (NBL2) or MDCK 33016, deposit number DSM ACC 2219 as described in WO97/37001), cat, and rodent (e.g. hamster cells such as BHK21-F, HKCC cells, or Chinese hamster ovary cells (CHO cells)), and may be obtained from a wide variety of developmental stages, including for example, adult, neonatal, fetal, and embryo. In certain embodiments, the cells are immortalized (e.g. PERC.6 cells, as described in WO 01/38362 and WO 02/40665, and as deposited under ECACC deposit number 96022940). In preferred embodiments, mammalian cells are utilized, and may be selected from and/or derived from one or more of the following non-limiting cell types: fibroblast cells (e.g. dermal, lung), endothelial cells (e.g. aortic, coronary, pulmonary, vascular, dermal microvascular, umbilical), hepatocytes, keratinocytes, immune cells (e.g. T cell, B cell, macrophage, NK, dendritic), mammary cells (e.g. epithelial), smooth muscle cells (e.g. vascular, aortic, coronary, arterial, uterine, bronchial, cervical, retinal pericytes), melanocytes, neural cells (e.g. astrocytes), prostate cells (e.g. epithelial, smooth muscle), renal cells (e.g. epithelial, mesangial, proximal tubule), skeletal cells (e.g. chondrocyte, osteoclast, osteoblast), muscle cells (e.g. myoblast, skeletal, smooth, bronchial), liver cells, retinoblasts, and stromal cells. WO 97/37000 and WO 97/37001 describe the production of animal cells and cell lines that are capable of growth in suspension and in serum free media and are useful in the production and replication of viruses. In one embodiment, the cells used for growing the at least one virus are Vero cells.

Culture conditions for the above cell types are known and described in a variety of publications. Alternatively, culture medium, supplements, and conditions may be purchased commercially, such as for example, described in the catalog and additional literature of Cambrex Bioproducts (East Rutherford, N.J.).

In certain embodiments, the cells used in the methods described herein are cultured in serum free and/or protein free media. A medium is referred to as a serum-free medium in the context of the present disclosure, if it does not contain any additives from serum of human or animal origin. Protein-free is understood to mean cultures in which multiplication of the cells occurs with exclusion of proteins, growth factors, other protein additives and non-serum proteins, but can optionally include proteins such as trypsin or other proteases that may be necessary for viral growth. The cells growing in such cultures naturally contain proteins themselves.

Known serum-free media include Iscove's medium, Ultra-CHO medium (BioWhittaker) or EX-CELL (JRH Bioscience). Ordinary serum-containing media include Eagle's Basal Medium (BME) or Minimum Essential Medium (MEM) (Eagle, Science, 130, 432 (1959)) or Dulbecco's Modified Eagle Medium (DMEM or EDM), which are ordinarily used with up to 10% fetal calf serum or similar additives. Optionally, Minimum Essential Medium (MEM) (Eagle, Science, 130, 432 (1959)) or Dulbecco's Modified Eagle Medium (DMEM or EDM) may be used without any serum containing supplement. Protein-free media like PF-CHO (JHR Bioscience), chemically-defined media like ProCHO 4CDM (BioWhittaker) or SMIF 7 (Gibco/BRL Life Technologies) and mitogenic peptides like Primactone, Pepticase or HyPep™ (all from Quest International) or lactalbumin hydrolysate (Gibco and other manufacturers) are also adequately known in the prior art. The media additives based on plant hydrolysates have the special advantage that contamination with viruses, mycoplasma or unknown infectious agents can be excluded.

Cell culture conditions (temperature, cell density, pH value, etc.) are variable over a very wide range owing to the suitability of the cell line employed according to the present disclosure and can be adapted to the requirements of particular viral strains.

The method for propagating virus in cultured cells generally includes the steps of inoculating the cultured cells with the strain to be cultured, cultivating the infected cells for a desired time period for virus propagation, such as for example as determined by virus titer or antigen expression (e.g. between 24 and 168 hours after inoculation) and collecting the propagated virus. In some embodiments, the virus is collected via plaque purification. The cultured cells are inoculated with a virus (measured by PFU or TCID50) to cell ratio of 1:500 to 1:1, preferably 1:100 to 1:5. The virus is added to a suspension of the cells or is applied to a monolayer of the cells, and the virus is absorbed on the cells for at least 10 minutes, at least 20 minutes, at least 30 minutes, at least 40 minutes, at least 50 minutes, at least 60 minutes but usually less than 300 minutes at 25° C. to 40° C., preferably 28° C. to 38° C. The infected cell culture (e.g. monolayers) may be removed either by harvesting the supernatant (free of cells), freeze-thawing or by enzymatic action to increase the viral content of the harvested culture supernatants. The harvested fluids are then either inactivated or stored frozen. Cultured cells may be infected at a multiplicity of infection (“MOI”) of about 0.0001 to 10, preferably 0.002 to 5, more preferably to 0.001 to 2. Still more preferably, the cells are infected at an MOI of about 0.01. During infection the ratio of culture medium to the area of the cell culture vessel may be lower than during the culture of the cells. Keeping this ratio low maximizes the likelihood that the virus will infect the cells. The supernatant of the infected cells may be harvested from 30 to 60 hours post infection, or 3 to 10 days post infection. In certain preferred embodiments, the supernatant of the infected cells is harvested 3 to 7 days post infection. More preferably, the supernatant of the infected cells is harvested 3 to 5 days post infection. In some embodiments, proteases (e.g., trypsin) may be added during cell culture to allow viral release, and the proteases may be added at any suitable stage during the culture. Alternatively, in certain embodiments, the supernatant of infected cell cultures may be harvested and the virus may be isolated or otherwise purified from the supernatant.

The viral inoculum and the viral culture are preferably free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses (WO 2006/027698).

Where virus has been grown on a cell line then it is standard practice to minimize the amount of residual cell line DNA in the final liquid inactivated virus composition, in order to minimize any oncogenic activity of the host cell DNA. Contaminating DNA can be removed during liquid inactivated virus composition preparation using standard purification procedures e.g. chromatography, etc. Removal of residual host cell DNA can be enhanced by nuclease treatment e.g. by using a DNase. A convenient method for reducing host cell DNA contamination disclosed in references (Lundblad (2001) Biotechnology and Applied Biochemistry 34:195-197, Guidance for Industry: Bioanalytical Method Validation. U.S. Department of Health and Human Services Food and Drug Administration Center for Drug Evaluation and Research (CDER) Center for Veterinary Medicine (CVM). May 2001.) involves a two-step treatment, first using a DNase (e.g. Benzonase), which may be used during viral growth, and then a cationic detergent (e.g. CTAB), which may be used during virion disruption. Removal by β-propiolactone treatment can also be used. In one embodiment, the contaminating DNA is removed by benzonase treatment of the culture supernatant.

### Production of Antigens

The Zika virus may be produced and/or purified or otherwise isolated by any suitable method known in the art. In one embodiment, the antigen of the present disclosure is a purified inactivated whole Zika virus.

In some embodiments, inactivated viruses can be produced as described in the above section entitled “Production of Inactivated Virus Compositions.”

In certain embodiments, the Zika virus of the present disclosure may be produced by culturing a non-human cell. Cell lines suitable for production of Zika virus of the present disclosure may include insect cells (e.g., any of the mosquito cells described herein). Cell lines suitable for production of Zika virus of the present disclosure may also be cells of mammalian origin, and include, but are not limited to: VERO cells (from monkey kidneys), horse, cow (e.g. MDBK cells), sheep, dog (e.g. MDCK cells from dog kidneys, ATCC CCL34 MDCK (NBL2) or MDCK 33016, deposit number DSM ACC 2219 as described in WO 97/37001), cat, and rodent (e.g. hamster cells such as BHK21-F, HKCC cells, or Chinese hamster ovary cells (CHO cells)), and may be obtained from a wide variety of developmental stages, including for example, adult, neonatal, fetal, and embryo. In certain embodiments, the cells are immortalized (e.g. PERC.6 cells, as described in WO 01/38362 and WO 02/40665, and as deposited under ECACC deposit number 96022940). In preferred embodiments, mammalian cells are utilized, and may be selected from and/or derived from one or more of the following non-limiting cell types: fibroblast cells (e.g. dermal, lung), endothelial cells (e.g. aortic, coronary, pulmonary, vascular, dermal microvascular, umbilical), hepatocytes, keratinocytes, immune cells (e.g. T cell, B cell, macrophage, NK, dendritic), mammary cells (e.g. epithelial), smooth muscle cells (e.g. vascular, aortic, coronary, arterial, uterine, bronchial, cervical, retinal pericytes), melanocytes, neural cells (e.g. astrocytes), prostate cells (e.g. epithelial, smooth muscle), renal cells (e.g. epithelial, mesangial, proximal tubule), skeletal cells (e.g. chondrocyte, osteoclast, osteoblast), muscle cells (e.g. myoblast, skeletal, smooth, bronchial), liver cells, retinoblasts, and stromal cells. WO 97/37000 and WO 97/37001 describe production of animal cells and cell lines that are capable of growth in suspension and in serum free media and are useful in the production of viral antigens. In certain embodiments, the non-human cell is cultured in serum-free media. In certain embodiments, the Zika virus of the present disclosure may be produced by culturing Vero cells.

### Virus Inactivation

The liquid inactivated virus composition according to the present invention comprises an inactivated whole Zika virus.

Methods of inactivating or killing viruses to destroy their ability to infect mammalian cells, but do not destroy the secondary, tertiary or quaternary structure and immunogenic epitopes of the virus are known in the art. Such methods include both chemical and physical means. Suitable means for inactivating a virus include, without limitation, treatment with an effective amount of one or more agents selected from detergents, formalin (also referred to herein as “formaldehyde”), hydrogen peroxide, beta-propiolactone (BPL), binary ethylamine (BEI), acetyl ethyleneimine, heat, electromagnetic radiation, x-ray radiation, gamma radiation, ultraviolet radiation (UV radiation), UV-A radiation, UV-B radiation, UV-C radiation, methylene blue, psoralen, carboxyfullerene (C₆₀), hydrogen peroxide and any combination of any thereof. As already mentioned above, for the purpose of the present application the terms “formalin” and “formaldehyde” are used interchangeably. When reference is made herein to a concentration of formaldehyde, it refers to the concentration of formaldehyde and not to the concentration of formalin. Accordingly, a “formaldehyde concentration of 0.01% (w/v)” refers to 0.01% (w/v) formaldehyde, and no further correction of this concentration for the formaldehyde concentration in the formalin stock solution (which typically contains 37% formaldehyde by mass) has to be made. For example, such a formaldehyde concentration in the virus preparation can be obtained by diluting formalin to a working solution having a formaldehyde content of 1.85% (w/v), which is then further diluted to the required concentration when it is mixed with the virus preparation such as the Zika virus preparation.

In certain embodiments of the present disclosure the at least one (Zika) virus is chemically inactivated. Agents for chemical inactivation and methods of chemical inactivation are well known in the art and described herein. In some embodiments, the at least one virus is chemically inactivated with one or more of BPL, hydrogen peroxide, formalin, or BEI. In certain embodiments where the at least one virus is chemically inactivated with BPL, the virus may contain one or more modifications. In some embodiments, the one or more modifications may include a modified nucleic acid. In some embodiments, the modified nucleic acid is an alkylated nucleic acid. In other embodiments, the one or more modifications may include a modified polypeptide. In some embodiments, the modified polypeptide contains a modified amino acid residue including one or more of a modified cysteine, methionine, histidine, aspartic acid, glutamic acid, tyrosine, lysine, serine and threonine.

In certain embodiments, the at least one (Zika) virus is inactivated with formaldehyde.

In certain embodiments where the at least one virus is chemically inactivated with formalin (formaldehyde), the inactivated virus may contain one or more modifications. In some embodiments, the one or more modifications may include a modified polypeptide. In some embodiments, the one or more modifications may include a cross-linked polypeptide. In some embodiments where the at least one virus is chemically inactivated with formalin, the liquid inactivated virus composition further includes formalin. In certain embodiments where the at least one virus is chemically inactivated with BEI, the virus may contain one or more modifications. In some embodiments, the one or more modifications may include a modified nucleic acid. In some embodiments, the modified nucleic acid is an alkylated nucleic acid.

In some embodiments, where the at least one virus is chemically inactivated with formalin, any residual unreacted formalin may be neutralized with sodium metabisulfite, may be dialyzed out, and/or may be buffer exchanged to remove the residual unreacted formalin. In some embodiments, the sodium metabisulfite is added in excess. In some embodiments, the solutions may be mixed using a mixer, such as an in-line static mixer, and subsequently filtered or further purified (e.g., using a cross flow filtrations system).

In some embodiments, the formaldehyde concentration is 0.005% (w/v) to 0.02% (w/v). In some embodiments, the formaldehyde concentration is 0.0075% (w/v) to 0.015% (w/v). In some embodiments, the formaldehyde concentration is 0.01% (w/v).

In some embodiments, the Zika virus is an inactivated whole virus obtained/obtainable by a method wherein the Zika virus is treated with formaldehyde in an amount that ranges from about 0.001% w/v to about 3.0% w/v for 5 to 15 days at a temperature that ranges from about 15° C. to about 37° C. In certain such embodiments, the Zika virus is an inactivated whole virus obtained/obtainable by treating a whole live Zika virus with 0.005% to 0.02% w/v of formaldehyde. In certain such embodiments, the Zika virus is an inactivated whole virus obtained/obtainable by treating a whole live Zika virus with less than 0.015% w/v of formaldehyde.

In certain embodiments, the inactivated whole Zika virus is considered to be obtainable/obtained from a method wherein the Zika virus is treated with formaldehyde in an amount that ranges from about 0.02% w/v for 14 days at a temperature of 22° C. In some embodiments, an inactivated whole Zika virus preparation is considered to be obtainable/obtained from a method wherein the Zika virus is treated with formaldehyde in an amount of about 0.01% w/v for 10 days at a temperature of 22° C.

### Zika Virus Purity

The purity of the Zika virus can be determined by size exclusion chromatography. Certain embodiments of the present disclosure relate to inactivated virus compositions comprising an inactivated whole Zika virus that is at least 85% pure as determined by the main peak of the Zika virus in the size exclusion chromatography being more than 85% of the total area under the curve. In certain such embodiments, the Zika virus may be 90% pure as determined by the main peak of the Zika virus in the size exclusion chromatography being more than 90°/% of the total area under the curve. In certain such embodiments, the Zika virus may be 95% pure as determined by the main peak of the Zika virus in the size exclusion chromatography being more than 95% of the total area under the curve.

### Use

In a certain embodiment, the present invention relates to the use of an inactivated virus composition comprising:
a) an inactivated whole Zika virus,
b) at least one pharmaceutically acceptable buffer with a concentration of at least about 6.5 mM, and
c) optionally a polyol,
   wherein the inactivated virus composition does not contain an adjuvant selected from aluminum salts and said at least one pharmaceutically acceptable buffer does not comprise phosphate ions, for stabilizing the inactivated whole Zika virus.

In certain embodiments, the present invention relates to the use of an inactivated virus composition in accordance with the present invention (as described above) for stabilizing an inactivated whole Zika virus.

In certain such embodiments, the present invention relates to the use of the inactivated virus composition for stabilizing the inactivated whole Zika virus during storage at 5±3° C. for at least 10 days.

In certain embodiments, the present invention relates to the use of the inactivated virus composition for stabilizing the inactivated whole Zika virus during storage at −80° C. for at least 10 days. In certain such embodiments, the present invention relates to the use of the inactivated virus composition for stabilizing the inactivated whole Zika virus during storage at −80° C. for at least 6 months. In certain such embodiments, the present invention relates to the use of the inactivated virus composition for stabilizing the inactivated whole Zika virus during storage at −80° C. for at least 12 months.

In certain such embodiments, the present invention relates to the use of the inactivated virus composition for stabilizing the inactivated whole Zika virus during one or multiple freeze thaw cycles, such as at least 4 freeze thaw cycles.

### Method of Manufacture of the Inactivated Virus Composition

In certain embodiments, the present invention relates to a method of preparing an inactivated virus composition comprising:
a) an inactivated whole Zika virus;
b) a pharmaceutically acceptable buffer, wherein the said buffer is not phosphate buffer and wherein the concentration of said buffer is at least 6.5 mM; and
c) optionally a polyol;
   wherein the inactivated virus composition does not contain an adjuvant selected from aluminum salts; the method comprising the following steps:
Step 1. isolating a Zika virus preparation supernatants obtained from one or more non-human cells,
Step 2. purifying the Zika virus preparation;
Step 3. inactivating the virus preparation;
Step 4. transferring the Zika virus preparation into a pharmaceutically acceptable buffer to obtain the Zika virus drug substance.

In some embodiments, the cells used in step 1 are non-human cells. Suitable non-human mammalian cells include, but are not limited to, VERO cells, LLC-MK2 cells, MDBK cells, MDCK cells, ATCC CCL34 MDCK (NBL2) cells, MDCK 33016 (deposit number DSM ACC 2219 as described in WO97/37001) cells, BHK21-F cells, HKCC cells, and Chinese hamster ovary cells (CHO cells). In some embodiments, the mammalian cells are Vero cells.

In step 2, any method of purifying a virus preparation known in the art may be employed to isolate the Zika virus, including, without limitation, using cross flow filtration (CFF), multimodal chromatography, size exclusion chromatography, cation exchange chromatography, and/or anion exchange chromatography. In some embodiments, the virus preparation is isolated by cross flow filtration (CFF). In some embodiments, the virus preparation is purified to a high degree in an amount that is about 70%, about 75%, about 80%, about 85%, about 90%, about 91%, about 92%, about 93%, about 94%, about 95% about 96%, about 97%, about 98%, about 99%, or more.

In step 3, the Zika virus preparation may be inactivated by being treated with 0.005 to 0.02% (w/v) formalin for eight to twelve days at a temperature of 15° C. to 30° C. In some embodiments, the Zika virus preparation is treated with 0.005 to 0.02% (w/v) formalin for nine to eleven days at a temperature of 15° C. to 30° C. In some embodiments, the Zika virus preparation is treated with 0.005 to 0.02% (w/v) formalin for ten days at a temperature of 15° C. to 30° C. In some embodiments, the Zika virus preparation is treated with 0.008 to 0.015% (w/v) formalin for eight to twelve days at a temperature of 15° C. to 30° C. In some embodiments, the Zika virus preparation is treated with 0.008 to 0.015% (w/v) formalin for nine to eleven days at a temperature of 15° C. to 30° C. In some embodiments, the Zika virus preparation is treated with 0.008 to 0.015% (w/v) formalin for ten days at a temperature of 15° C. to 30° C. In some embodiments, the Zika virus preparation is treated with 0.01% (w/v) formalin for eight to twelve days at a temperature of 15° C. to 30° C. In some embodiments, the Zika virus preparation is treated with 0.01% (w/v) formalin for nine to eleven days at a temperature of 15° C. to 30° C. In some embodiments, the Zika virus preparation is treated with 0.01% (w/v) formalin for ten days at a temperature of 15° C. to 30° C.

In some embodiments, step 3 further involves neutralizing unreacted formalin with an effective amount of sodium metabisulfite.

In certain embodiments, the present invention also relates to a product (such as an inactivated virus composition) obtainable by the method described above.

### Zika Virus Vaccine

The inactivated virus compositions according to the present invention generally refer to intermediate compositions used in the manufacture of vaccines. A certain further aspect of the present invention relates to liquid vaccines (or compositions suitable for use in the treatment or prevention of a disease or condition, in particular for compositions suitable for use in the treatment or prevention of Zika virus infection), obtained/obtainable from the inactivated virus compositions described above. The vaccine contains adjuvants and may have buffer and excipient concentrations different from the inactivated virus composition.

In certain such embodiments, the present invention relates to a liquid vaccine comprising:
a) the inactivated virus composition according to any one of the preceding claims, and
b) an adjuvant, such as aluminum hydroxide.

In certain such embodiments, the present invention relates to a liquid vaccine comprising an inactivated Zika virus, wherein the concentration of sodium chloride in the liquid vaccine is from about 50 mM to about 200 mM, or from about 50 mM to about 150 mM, such as about 84 mM. In certain such embodiments, the present invention relates to a liquid vaccine, wherein the liquid vaccine comprises from about 8.5 mM to about 80 mM Tris and from about 50 mM to about 150 mM NaCl, and wherein the pH of the liquid vaccine is from about pH 7.0 to about pH 8.0, when measured at room temperature.

In certain such embodiments, the concentration of Tris in the liquid vaccine is from about 9 mM to about 80 mM, or from about 9 mM to about 60 mM, or from 9 mM to about 30 mM, or from about 9 mM to about 11 mM or about 10 mM.

In certain embodiments, the present invention relates to a liquid vaccine wherein the liquid vaccine comprises from about 0.4% (w/v) to 4.7% (w/v) sucrose.

In certain embodiments, the osmolality of the liquid vaccine is about 300±50 mOsm/kg. Osmolality is determined via freezing point depression in an Advanced Instruments OsmoPRO® Multi-Sample Micro-Osmometer (Fisher Scientific, Pittsburgh, Pa.), following the manufacturer's instructions and using their calibration and reference solutions.

### Adjuvants

The vaccines according to the present invention comprise one or more antigens from at least one Zika virus, in combination with one or more adjuvants.

Various methods of achieving an adjuvant effect for vaccines are known and may be used in conjunction with the Zika virus vaccines disclosed herein. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9.

Exemplary adjuvants may include, but are not limited to, aluminum salts, calcium phosphate, toll-like receptor (TLR) agonists, monophosphoryl lipid A (MLA), MLA derivatives, synthetic lipid A, lipid A mimetics or analogs, cytokines, saponins, muramyl dipeptide (MDP) derivatives, CpG oligos, lipopolysaccharide (LPS) of gram-negative bacteria, polyphosphazenes, emulsions (oil emulsions), chitosan, vitamin D, stearyl or octadecyl tyrosine, virosomes, cochleates, poly(lactide-co-glycolides) (PLG) microparticles, poloxamer particles, microparticles, liposomes, Complete Freund's Adjuvant (CFA), and Incomplete Freund's Adjuvant (IFA). In some embodiments, the adjuvant is an aluminum salt.

In some embodiments, the adjuvant includes at least one of alum (such as aluminum hydroxide), aluminum phosphate, aluminum oxide hydroxide, aluminum hydroxide, precipitated aluminum hydroxide, potassium aluminum sulfate, and gel-like aluminum hydroxide such as, e.g. Alhydrogel 85. Hereinafter, aluminum oxide hydroxide, aluminum hydroxide and precipitated and/or gel-like aluminum hydroxide in a pharmaceutically acceptable form, in particular for use as adjuvants, are also collectively referred to as “aluminum hydroxide”. In some embodiments, aluminum salt adjuvants of the present disclosure have been found to increase adsorption of the antigens of the Zika virus vaccines of the present disclosure. Accordingly, in some embodiments, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the antigen is adsorbed to the aluminum salt adjuvant.

Certain embodiments of the present disclosure include a method for preparing an adjuvanted Zika virus vaccine, which involves (a) mixing the vaccine with an aluminum salt adjuvant, with the vaccine including one or more antigens from at least one Zika virus described herein and (b) incubating the mixture under suitable conditions for a period of time that ranges from about 1 hour to about 24 hours (e.g., about 16 hours to about 24 hours), with at least about 75%, at least about 80%, at least about 85%, at least about 90%, at least about 91%, at least about 92° %, at least about 93%, at least about 94%, at least about 95%, at least about 96%, at least about 97%, at least about 98%, at least about 99%, or about 100% of the antigen adsorbed to the aluminum salt adjuvant. In certain embodiments of the method, the at least one Zika virus is a Zika virus comprising a non-human cell adaptation mutation (e.g., a non-human cell adaptation mutation in protein NS1 such as a Trp98Gly mutation). In some embodiments, the at least one Zika virus is a purified inactivated whole Zika virus comprising a Trp98Gly mutation at position 98 of SEQ ID NO: 1, or at a position corresponding to position 98 of SEQ ID NO: 1, wherein the Zika virus is derived from strain PRVABC59. In some embodiments, the Zika virus is a purified inactivated whole Zika virus comprising a Trp98Gly mutation at position 98 of SEQ ID NO: 1, or at a position corresponding to position 98 of SEQ ID NO: 1, wherein the Zika virus is derived from strain PRVABC59 comprising the genomic sequence according to SEQ ID NO: 2.

Without wishing to be bound by any theory, absorbing the Zika virus antigen (inactivated whole Zika virus) to an aluminum salt (such as e.g. aluminum hydroxide/alum) may enhance the stability of the Zika virus antigen.

In certain preferred embodiments, the adjuvant is aluminum hydroxide.

In certain embodiments, the present invention relates to a liquid vaccine comprising 100 μg/ml to 800 μg/ml aluminum hydroxide, or 200 μg/ml to 600 μg/ml aluminum hydroxide, or 300 μg/ml to 500 μg/ml aluminum hydroxide, or about 400 μg/ml aluminum hydroxide based on elemental aluminum.

In certain such embodiments, the present invention relates to a liquid vaccine, wherein the liquid vaccine comprises from about 8.5 mM to about 50 mM Tris and from about 50 mM to about 150 mM NaCl, and from about 300 μg/ml to about 500 μg/ml aluminum hydroxide based on elemental aluminum and wherein the pH of the liquid vaccine is from about pH 7.0 to about pH 8.0, when measured at room temperature.

### Dose

In certain embodiments, the present invention is directed to a unit does of the liquid vaccine according to the present invention.

In certain such embodiments, the unit does of the liquid vaccine comprises a dose of from about 1 μg to about 15 μg of the inactivated whole Zika virus. In certain such embodiments, the unit dose of vaccine comprises a dose of about 2 μg of inactivated whole Zika virus. In certain such embodiments, the unit dose of vaccine comprises a dose of about 5 μg of inactivated whole Zika virus. In certain such embodiments, the unit dose of vaccine comprises a dose of about 10 μg of inactivated whole Zika virus.

In certain embodiments, the unit dose of vaccine is provided as about 0.4 mL to about 0.8 mL of a pharmaceutically acceptable liquid.

In certain such embodiments, the unit does of the liquid vaccine comprises from about 100 μg to about 300 μg of aluminum hydroxide, such as about 200 μg of aluminum hydroxide, based on elemental aluminum. As is well-known to the skilled person, the phrase “based on elemental aluminum” refers to the way the aluminum content of a vaccine formulation is specified. The varying amount of water and the complex stoichiometry of (hydrated) aluminum hydroxide, aluminum oxide hydroxide and related aluminum compounds necessitate a standardized way to indicate the aluminum content of a composition. Towards this, typically the amount of aluminum ions, expressed as “elemental aluminum”, is given. Accordingly, for example a composition said to contain “100 μg/ml aluminum hydroxide based on elemental aluminum” (or, as often found in short form, a composition said to contain “100 μg/ml aluminum hydroxide”) contains 100 μg/ml aluminum ions.

### Method of Treatment

In certain embodiments, the present invention relates to a method of treating or preventing in particular preventing Zika virus infection in a human subject in need thereof, comprising administering to the subject a unit dose of the vaccine in accordance with the present invention.

In certain embodiments, the present invention relates to a method of treating or preventing in particular preventing Zika virus infection in a human subject population in need thereof, comprising administering to individual human subjects of said human subject population a unit dose of the vaccine in accordance with the present invention.

In certain embodiments, the present invention relates a unit dose of the vaccine in accordance with the present invention, for use in treating or preventing, in particular preventing Zika virus infection in a human subject in need thereof.

In certain embodiments, the present invention relates to the use of a unit dose of the vaccine in accordance with the present invention in the manufacture of a medicament for preventing Zika virus infection in a human subject in need thereof.

In some embodiments, the present disclosure relates to methods for inducing an immune response to Zika virus in a subject in need thereof by administering to the subject a therapeutically effective amount of the vaccine according to the present invention. In some embodiments, the administering step induces a protective immune response against Zika virus in a subject.

In certain such embodiments, the subject is a female subject. In some embodiments, the subject is pregnant or intends to become pregnant.

The methods of the present disclosure include administration of a therapeutically effective amount or an immunogenic amount of the Zika virus vaccines of the present disclosure. A therapeutically effective amount or an immunogenic amount may be an amount of the vaccines of the present disclosure that will induce a protective immunological response in the uninfected, infected or unexposed subject to which it is administered. Such a response will generally result in the development in the subject of a secretory, cellular and/or antibody-mediated immune response to the vaccine. Usually, such a response includes, but is not limited to one or more of the following effects; the production of antibodies from any of the immunological classes, such as immunoglobulins A, D, E, G or M; the proliferation of B and T lymphocytes; the provision of activation, growth and differentiation signals to immunological cells; expansion of helper T cell, suppressor T cell, and/or cytotoxic T cell.

Preferably, the therapeutically effective amount or immunogenic amount is sufficient to bring about treatment or prevention of disease symptoms. The exact amount necessary will vary depending on the subject being treated; the age and general condition of the subject to be treated; the capacity of the subject's immune system to synthesize antibodies; the degree of protection desired; the severity of the condition being treated; the particular Zika virus antigen selected and its mode of administration, among other factors. An appropriate therapeutically effective amount or immunogenic amount can be readily determined by one of skill in the art. A therapeutically effective amount or immunogenic amount will fall in a relatively broad range that can be determined through routine trials.

Typically, the vaccines of the present disclosure are prepared as injectables either as liquid solutions or suspensions.

Vaccines may be conventionally administered parenterally, by injection, for example, either subcutaneously, transcutaneously, intradermally, subdermally or intramuscularly. Additional formulations which are suitable for other modes of administration include suppositories and, in some cases, oral, peroral, intranasal, buccal, sublingual, intraperitoneal, intravaginal, anal and intracranial formulations. For suppositories, traditional binders and carriers may include, for example, polyalkalene glycols or triglycerides; such suppositories may be formed from mixtures containing the active ingredient in the range of 0.5% to 10%, or even 1-2%. In certain embodiments, a low melting wax, such as a mixture of fatty acid glycerides or cocoa butter is first melted and the Zika virus vaccine described herein is dispersed homogeneously, for example, by stirring. The molten homogeneous mixture is then poured into conveniently sized molds and allowed to cool and to solidify.

The vaccines of the present disclosure may be administered in a manner compatible with the dosage formulation, and in such amounts as will be therapeutically effective and immunogenic. The quantity to be administered depends on the subject to be treated, including, e.g., the capacity of the individual's immune system to mount an immune response, and the degree of protection desired. Suitable dosage ranges may include, for example, from about 0.1 μg to about 100 μg of the purified inactivated whole Zika virus

Suitable regimens for initial administration and booster shots are also variable but are typified by an initial administration followed by subsequent inoculations or other administrations.

### Method of Manufacture of the Vaccine

In certain embodiments, the present invention also relates to a method of preparing a liquid vaccine, the method comprising the following steps:
Step 1. providing an inactivated virus composition in accordance with the present invention,
Step 2. adding an adjuvant preferably an aluminum salt and optionally a further pharmaceutically acceptable buffered liquid to the inactivated virus composition.

In certain such embodiments, in step 2 the further pharmaceutically acceptable buffered liquid comprises the same buffer as the buffer with the highest concentration in the inactivated virus composition.

In certain embodiments, the present invention relates to a product obtainable by the methods described above.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Clonal Zika Virus Strain Generation

This example describes the production of Zika virus (ZIKAV) strains with a known research history.

### Materials and Methods

### Vero Cell Maintenance

One vial of WHO Vero 10-87 cells was rapidly thawed in a water bath and directly inoculated into 19 mL of pre-warmed DMEM (Dulbecco's modified minimal essential medium) containing penicillin-streptomycin, L-glutamine 40 mM, and 10% FBS in a T-75 cm² flask at 36° C.+/2° C., at 5% CO₂. Cells were allowed to grow to confluency and subcultured using TryplE. This flask was expanded to two T-185 cm² flasks, grown to confluency and subcultured to 31 xT-185 cm² flasks and grown until the cells reached 100% confluency. Cells were harvested by trypsinization, centrifuged at 800×g for 10 minutes, and resuspended in DMEM containing 10% FBS and 10% DMSO at a concentration of 1.9×10⁷ cells/mL. One vial of the Vero cells was rapidly thawed and resuscitated as described above into a T-75 cm² flask. These were subcultured twice to produce a cell bank in 13×T-185 cm² flasks. After trypsinization, the cells were centrifuged at 800×g and resuspended in freezing media (DMEM containing 10% FBS, and 10% DMSO) at a concentration of 4.68×10⁵ cells/mL. This cell bank was aliquoted into cryovials.

The Vero cells were grown and maintained in DMEM containing penicillin-streptomycin, L-glutamine and 10% FBS (cDMEM-10%-FBS). TryplExpress was used to maintain and trypsinize cells. Two days before viral adsorption, 6-well plates were seeded with 4-5×10⁵ cells/well in 3 mL of cDMEM-10%-FBS or 7×10⁵ cells in T-25 cm² flasks in 5 mL cDMEM-10%-FBS, or 1×10⁴ cells/well in 96-well plates in 0.1 mL cDMEM-10%-FBS. Incubators were monitored daily to maintain indicated temperatures. The Vero cell lines were stored in liquid nitrogen.

### Plaque Assay

Viral titers were determined by plaque titration in freshly confluent monolayers of Vero cells grown in 6-well plates. Frozen aliquots were thawed and ten-fold dilution series of the aliquots were made in cDMEM-0%-FBS in 96-well plates. The diluted viruses were maintained on ice prior to inoculation of the Vero cell monolayers. At the time of assay, the growth medium was aspirated from the 6-well plate, and 100 μL of each virus dilution was added to the wells. Virus was adsorbed for 60 min at 36° C.+2° C., at 5% CO₂, with frequent (every 10 min) rocking of the plates to prevent drying of the cell sheets. Following viral adsorption, 4 mL of a first agarose overlay (1×cDMEM-2%-FBS+0.8% agarose) maintained at 40-41° C. was added to each well. The agarose was allowed to solidify for 30 min at room temperature, and the plates were then incubated upside down for 4-6 days at 36° C.+/2° C., at 5% CO₂. Two mL of a second agarose overlay containing 160 μg/mL of neutral red vital dye was added on day 4. Plaques were visualized on days 5 and 6.

### Virus Quantification by TCID50 Assay

Viral titers were also determined by titration in freshly confluent monolayers of Vero cells grown in 96-well plates. Frozen aliquots were thawed and ten-fold dilution series of the aliquots were made in cDMEM-2%-FBS diluent in 96-well plates. The diluted viruses were maintained on ice prior to inoculation of the Vero cell monolayers. At the time of assay, the growth medium was aspirated from the 96-well plate, and 100 μL of each virus dilution was added to the wells. The plates were incubated for 5 days at 36° C.+/2° C., at 5% CO₂. The 50% Tissue Culture Infective Dose (TCID50) titer was calculated using the Reed/Muench calculator.

### Test Articles

Zika virus strain PRVABC59 (one 0.5 mL vial on dry ice) was received from the Centers for Disease Control and Prevention (CDC) Zika virus identification was confirmed through RT-PCR. The strain tested negative for Alphavirus and mycoplasma contamination by PCR. This information is summarized in Table 1.

TABLE 1
PRVABC59 strain information

| | Isolation | Patient | | | |
|---|---|---|---|---|---|
| Strain | Information | information | Prep info | Analyses | PFU |
| PRVABC59 | Human | None | Passage: | Sequencing | 6.7 log |
| (Asian) | serum; | provided | Vero(2)C6/36(1) | by ion | pfu/mL |
| | travel to | | Prep: Jan. 29, 2016 | torrent: | |
| | Puerto Rico | | Host: C6/36 | gene | |
| | in 2015 | | | accession | |
| | | | | #KU501215 | |
| | | | | PFU by | |
| | | | | plaque | |
| | | | | assay | |
| | | | | CI Identity by | |
| | | | | RT-PCR | |
| | | | | (-) For | |
| | | | | alphaviruses | |
| | | | | by PCR | |
| | | | | (-) for | |
| | | | | mycoplasma | |
| | | | | by ATCC | |
| | | | | and ABM | |
| | | | | PCR | |

### Sequencing

A QIAampViral RNA Mini Spin kit was used to extract RNA from stabilized virus harvests of each isolate according to manufacturer protocols. Extracted RNA from each isolate was used to create and amplify 6 cDNA fragments encompassing the entire Zika viral genome. Amplified cDNA fragments were analyzed for size and purity on a 1% Agarose/TBE gel and subsequently gel purified using a Qiagen Quick Gel Extraction Kit. An ABI 3130XL Genetic Analyzer sequencer was used to conduct automatic sequencing reactions. Lasergene SeqMan software was used to analyze sequencing data.

### Results

A ZIKAV strain with a known research history that was relevant to the current ZIKAV outbreak in the Americas was sought. For this reason, ZIKAV strain PRVABC59 was chosen. To generate a well-characterized virus adapted for growth in Vero cells, the ZIKAV PRVABC59 was first amplified in Vero cells (P1).

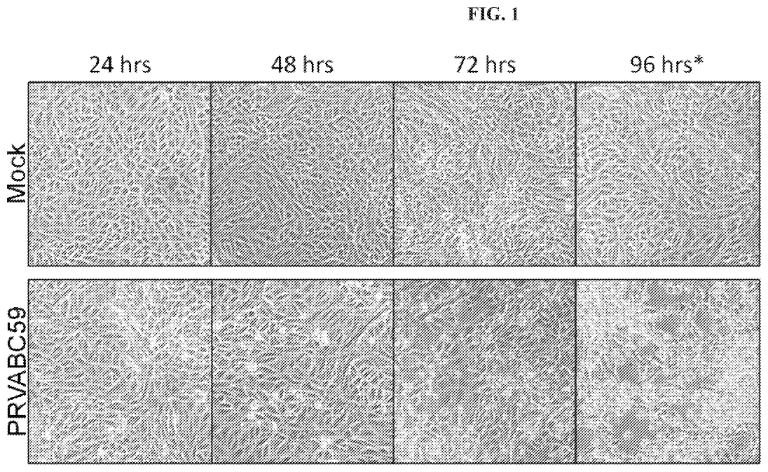

Flasks of Vero cells (T-175 cm²), 100% confluent, were infected at an MOI of 0.01 in 4 mL of cDMEM-0%-FBS. Virus was adsorbed to the monolayer for 60 minutes at 36° C.±2° C., at 5% CO₂, then 20 mL of cDMEM-0%-FBS was applied for viral amplification at 36° C.±2° C., at 5% CO₂. The cell layer was monitored daily for cytopathic effect (CPE) following inoculation (FIG. 1). The supernatant was harvested after 96 hours by collecting the media and clarifying by centrifugation (600×g, 4° C., 10 min). The harvest was stabilized by adding trehalose to a final concentration of 18% w/v. The bulk was aliquoted into 0.5 mL cryovials and stored at −80° C.

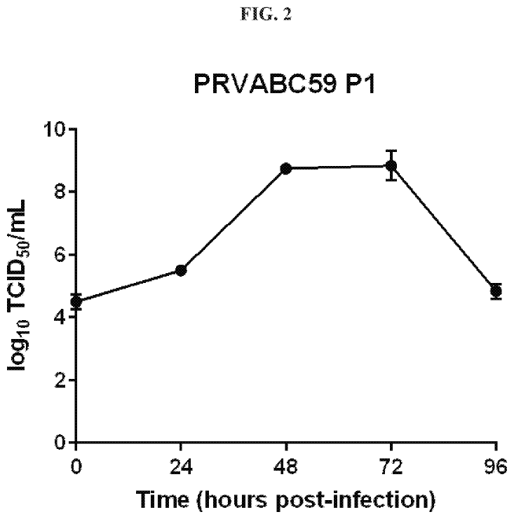

The stabilized P1 harvest was analyzed for the presence of infectious virus on Vero cell monolayers by a TCID50 assay. Growth kinetics were monitored by taking daily aliquots beginning on hour 0. Peak titer was reached by hour 72 (FIG. 2).

P1 material was plaque-purified by titrating the harvest from day 3 on 6-well monolayers of Vero cells. Plaques were visualized on day 6, and 10 plaques to be isolated were identified by drawing a circle around a distinct and separate plaque on the bottom of the plastic plate. Plaques were picked by extracting the plug of agarose using a sterile wide bore pipette while scraping the bottom of the well and rinsing with cDMEM-10%-FBS. The agarose plug was added to 0.5 mL of cDMEM-10%-FBS, vortexed, labeled as PRVABC59 P2a-j and placed in an incubator overnight at 36° C.±2° C., at 5% CO₂.

Three plaques (PRVABC59 P2a-c) were carried forward for additional purification. Each isolate was plated neat in duplicate onto a fresh 6-well monolayer of Vero cells. This P2/P3 transition was plaque purified, and labeled PRVABC59 P3a-j.

Six plaques (PRVABC59 P3a-f) were carried forward for a final round of purification. Each isolate was plated neat in duplicate onto a fresh 6-well monolayer of Vero cells. This P3/P4 transition was plaque purified, and labeled PRVABC59 P4a-j.

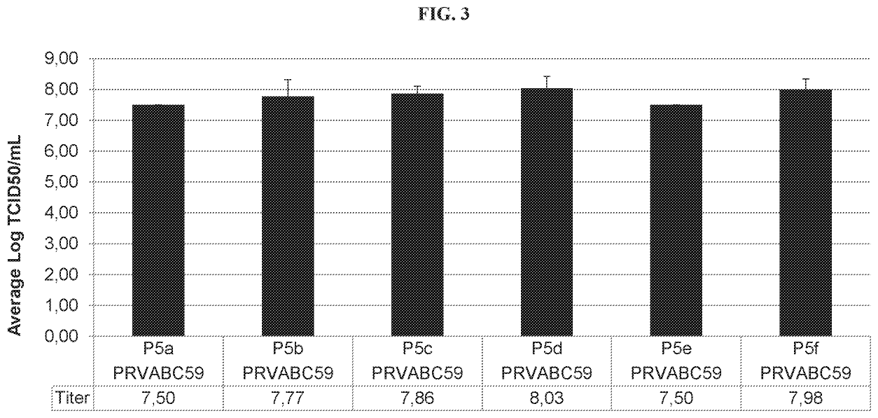

Six plaques (PRVABC59 P4a-f) from the P4 plaque purification were blind passaged on monolayers of Vero cells in T-25 cm² flasks. Each plaque pick was diluted in 2 mL cDMEM-0%-FBS—1 mL was adsorbed for 1 hour at 36° C.±2° C., at 5% CO₂; the other 1 mL was stabilized with trehalose (18% v/v final) and stored at <−60° C. Following virus adsorption, cDMEM-0%-FBS was added to each flask and allowed to grow at 36° C.±2° C., at 5% CO₂ for 4 days. Virus supernatants were harvested, clarified by centrifugation (600×g, 4C, 10 min), stabilized in 18% trehalose and aliquoted and stored at <−60° C. This P5 seed was tested by TCID50 for Zika virus potency (FIG. 3).

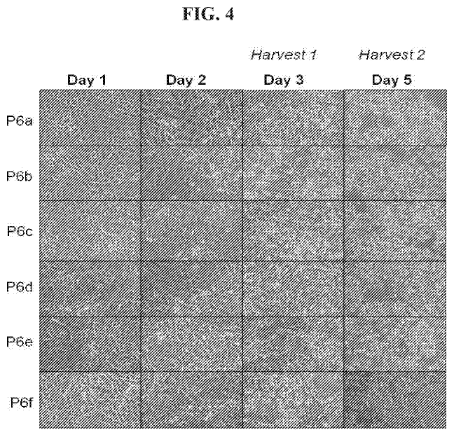

Confluent monolayers of T-175 cm² flasks of Vero cells were infected with each of the six clones of PRVABC59 (P5a-f) at an MOI of 0.01 in 4 mL cDMEM-0%-FBS. The virus was allowed to adsorb for 60 minutes at 36° C.+/2° C., at 5% CO₂, after which 20 mL of cDMEM-0%-FBS was added to each flask and allowed to grow at 36° C.+/2° C., at 5% CO₂. Vero cell monolayer health and CPE was monitored daily. Virus was harvested on days 3 and 5 as indicated (FIG. 4). The P6 strain harvests from days 3 and 5 were pooled, stabilized with 18% trehalose, aliquoted and stored<−60° C.

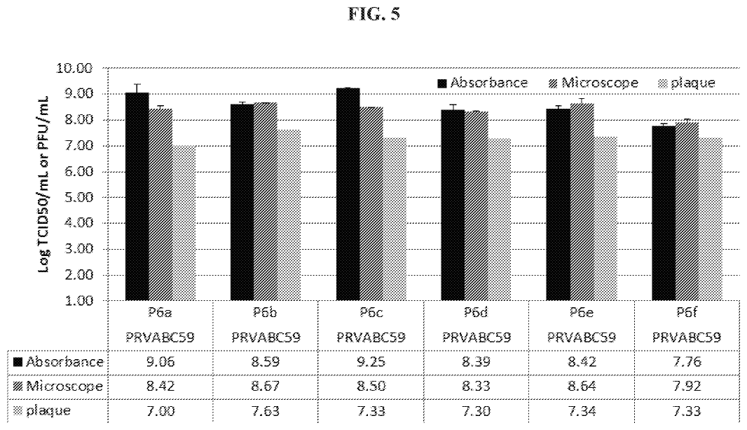

Each of the six clones of PRVABC59 (P6a-f) were tested for Zika virus in vitro potency (FIG. 5). The potency was determined by two different methods, TCID50 and plaque titration. The TCID50 was calculated by visual inspection of CPE (microscope) and by measuring the difference in absorbance (A560-A420) of the wells displaying CPE (yellow in color) compared with red (no CPE). The plates were read on a plate reader, and applied to the same calculator as the microscopically read-plates (absorbance). The values in TCID50 between the two scoring techniques are quite similar, while the values obtained by plaque titration are lower.

A summary of the generation of the P6 virus and characterization is shown in Table 2 below.

TABLE 2
Summary of virus passage and characterization for the
generation of clonal ZIKAV strains

| Passage | Seed production/purification | Characterization |
|---|---|---|
| P1 | Virus amplification in Vero | TGID50 titer |
| P2 | Amplify P1 by plaque titration; , Plaque | plaque purification |
| | purification of P1 | |
| P3 | Pick and passage plaques from P2 | plaque purification |
| | plaque assay; plaque purification of P2 | |
| P4 | Pick and passage plaques from P3 | plaque purification |
| | plaque assay; plaque purification of P3 | |
| P5 | Amplify P4 plaques (a-f) in Vero cells | TCID50 titer |
| P6 | Amplify P5 (a-f) virus in Vero cells | TCID50 titer, plaque |
| | | phenotype, genotype, |
| | | full genome sequencing, |
| | | growth kinetics |

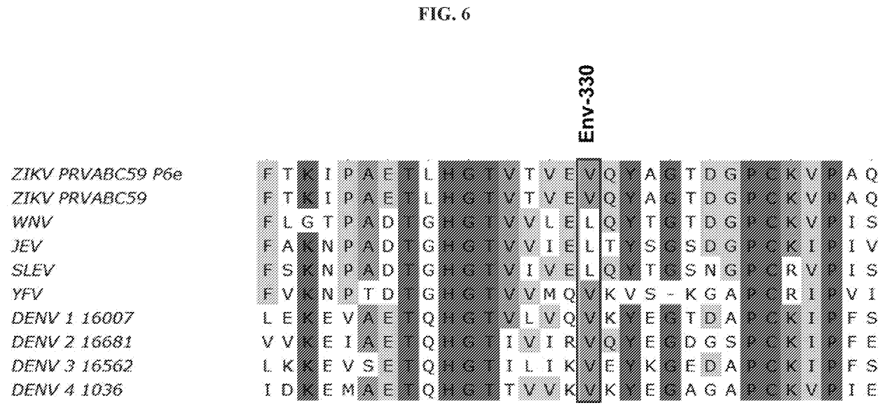

An isolated Zika virus clone that closely resembled the envelope glycoprotein sequence of the original isolate was sought, since the envelope protein of flaviviruses is the dominant immunogenic portion of the virus. PRVABC59 clones P6a, P6c, P6d and P6f contained a G→T mutation at nucleotide 990 in the envelope region (G990T), resulting in an amino acid mutation of Val→Leu at envelope residue 330, whereas the envelope gene of PRVABC59 clones P6b and P6e were identical relative to the reference strain (GenBank ref KU501215.1) (Table 3 and FIG. 6).

TABLE 3
Sequencing of PRVABC59 P6 clones
Envelope sequencing (reference gene from PRVABC59;
accession #KU501215)

| Strain | Nucleotide | Amino Acid | Mutation | Comments |
|---|---|---|---|---|
| PRVABC59 | Env-990: | Env-330: | Val/Leu | Mutation in 3 of 4 |
| P6a | G→T | Val330→Leu | | reads. |
| PRVABC59 | Env-1404: | Wild type | Wild type | Wild type relative |
| P6b | T→G silent | | | to reference. |
| PRVABC59 | Env-990: | Env-330: | Val/Leu | Mutation in 3 of 4 |
| P6c | G→T | Val330→Leu | | reads. |
| PRVABC59 | Env-990: | Env-330: | Val/Leu | Mutation in 2 of 2 |
| P6d | G→T | Val330→Leu | | reads. |
| PRVABC59 | Wild type | Wild type | Wild type | Wild type relative |
| P6e | | | | to reference. |
| PRVABC59 | Env-990: | Env-330: | Val/Leu | Mutation in 2 of 2 |
| P6f | G→T | Val330→Leu | | reads. 190 bp not |
| | | | | sequenced (aa |
| | | | | 421-484). |

Full genome sequencing (reference gene from PRVABC59;
accession #KU501215)

| | | | | |
|---|---|---|---|---|
| PRVABC59 | Env-1404 | Wild-type | Silent | Mutation in 2 of 2 |
| P6b | T→G | | | reads |
| | NS1-292 | NS1-98 | Trp/Gly | Mutation in 2 of 2 |
| | T→G | Trp98→Gly | | reads |
| PRVABC59 | NS1-292 | NS1-98 | Trp/Gly | Mutation in 2 of 2 |
| P6e | T→G | Trp98→Gly | | reads |

The two clones lacking mutations in the Zika envelope sequence were then subjected to full genome sequencing. Sequencing results are summarized in Table 3 above. Sequence analysis revealed a T→G substitution at nucleotide 292 in the NS1 region for both clones, resulting in a Trp→Gly mutation at NS1 residue 98. This mutation was also later confirmed through deep sequencing. The NS1 W98G mutation is located in the intertwined loop of the wing domain of ZIKAV NS1, which has been implicated in membrane association, interaction with envelope protein and potentially hexameric NS1 formation. While other tryptophan residues (W115, WI 118), are highly conserved across flaviviruses, W98 is not (FIG. 7). Interestingly, however, 1000% conservation of the W98 residue is observed across 11 different ZIKAV strains, including those from the African and Asian lineages. The identified mutations in each strain are summarized in Table 4.

TABLE 4
Summary of mutations identified in PRVABCS9 P6 clones

| | | |
|---|---|---|
| Clone | Nucleotide | Amino Acid |

Mutations identified in envelope

| | | |
|---|---|---|
| P6a | G9901 | V330L |
| P6b | T1404G | (silent) |
| P6e | G990T | V330L |
| P6d | G990T | V330L |
| P6e | none | none |
| P6f | G990T | V330L |

Additional mutations identified in genome

| | | |
|---|---|---|
| P6b | NS1-T292G | NS1-W98G |
| P6e | NS1-T292G | NS1-W98G |

Ref sequence: KU501215.1 (PRVABC59)

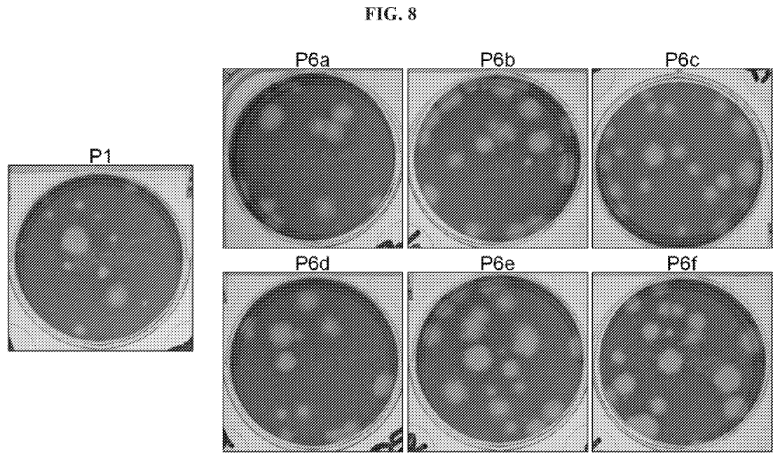

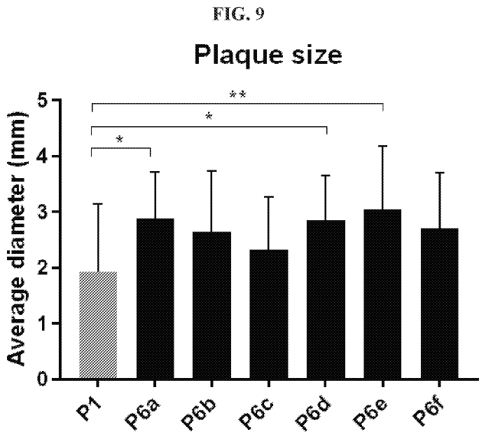

Phenotypic analysis of the ZIKAV PRVABC59 P6 stocks was conducted to characterize the ZIKAV clones. As illustrated in FIG. 8 and quantified in FIG. 9, each clonal isolate consisted of a relatively homogeneous population of large-sized plaques as compared to the P1 virus, which had a mixed population of large and small plaques. These data suggest the successful isolation of single ZIKAV clones.

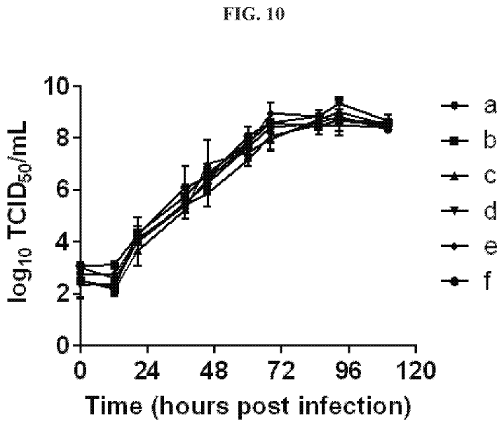

Next, growth kinetics analyses in Vero cells of the ZIKAV PRVABC59 P6 clones were analyzed. Vero cells were infected with 0.01 TCID50/cell of each ZIKAV P6 clones in serum free growth medium. Viral supernatant samples were taken daily and simultaneously assayed for infectious titer by TCID50 assay. For all P6 clones, peak titer occurred between day 3 and 4 (˜9.0 log₁₀ TCID50/mL). There was no significant difference in growth kinetics of the various P6 clones (FIG. 10).

Taken together, the results indicate that a Zika virus seed was successfully generated. This seed selection required understanding of growth history, kinetics, yield, genotype, and phenotype of the virus. Importantly, clonal isolation of the Zika virus strains allowed for the successful purification of the virus away from contaminating agents (e.g., adventitious agents that may be in the parental human isolate). Interestingly, three sequential plaque purifications succeeded in quickly selecting Vero-cell adapted virus (strains P6a-f), where these strains were able to replicate well in serum-free Vero cell cultures, with strain P6a, c, d, and f harboring a mutation in the viral envelope protein, while strains p6b and p6e obtained a mutation in the viral NS1 protein (with no modification to the viral envelope). Additionally, the Vero-adapted strains enabled efficient and reproducible growth and manufacture of subsequent viral passages propagated from these strains. Without wishing to be bound by theory, the Env-V330L mutation observed in strains P6a, c, d, and f may potentially be a result of in vitro adaptation, as a mutation at Env 330 was also observed upon passaging in Vero cells (Weger-Lucarelli et al. 2017. Journal of Virology). Because the envelope protein is the dominant immunogenic epitope of Zika virus, strains containing a Vero adaptive mutation in Env may negatively impact vaccine immunogenicity. Without wishing to be bound by theory, the adaptation mutation in protein NS1 appears not only to enhance viral replication, but may also reduce or otherwise inhibit the occurrence of undesirable mutations, such as in the envelope protein E (Env) of the Zika virus. In addition, NS1 may be known to bind to the Envelope protein during the life cycle of the virus. This mutation (NS1 W98G) may be implicated in changing the ability of the NS1 to associate, and possibly co-purify, with the virus during downstream processing. NS1 is also known to be immunogenic, and could be implicated in the immune response to the vaccine.

### Example 2: Completeness of Inactivation Assay to Determine Effectiveness of Inactivation

A double-infectivity assay also called completeness of inactivation (COI) assay was developed to determine the effectiveness of formaldehyde-inactivation (0.01% formaldehyde) and potential residual infectious viral activity of purified inactivated zika virus (PIZV) bulk drug substance (BDS).

Sample preparation: Four Purified Inactivated Zika Vaccine (PIZV) lots (Tox lots 1-4) of clone e as described above were manufactured by growth in Vero cells. Supernatants from 4 daily harvests (totaling about 4000 mL) were purified by chromatography followed by addition of formaldehyde to a final concentration of 0.01%. w/v. Inactivation was allowed to proceed for 10 days at 22° C. In Process Control (IPC) samples were removed on a daily basis from the bulk drug substance (BDS) during inactivation for characterization and analytics. The daily IPC samples were neutralized with sodium metabisulfite and dialysed into DMEM (viral growth media). The samples contain the purified inactivated Zika virus. On the final day of inactivation, the remaining volume of BDS samples was not neutralized, but was processed with TFF to remove formaldehyde and buffer exchanged into PBS.

Completeness of inactivation assay (COI): The COI assay was used for analysis of the effectiveness of inactivation in the daily IPC samples to understand the kinetics of inactivation, and the final BDS. For maximum sensitivity, two cell lines, Vero and C6/36, were initially utilized in this assay to detect potential live virus in the IPC and DS samples. When Zika virus infects Vero cells in the presence of growth medium containing phenol red, the by-products of cell death cause a drop in pH. Consequently, the media color changes from red/pink to yellow, indicative of this acidic shift in the media pH. This phenomenon is caused by the apoptosis and cytopathic effects (CPE), which refers to the observed changes in the cell structure of host cells that are caused by viral invasion, infection, and budding from the cells during viral replication. Ultimately, while both C6/36 mosquito and Vero cells are a permissive cell line for infection, Zika virus infection kills only Vero cells in vitro. Therefore, Vero cells were used as the indicator cell line for the assay. In contrast, C6/36 cells, which are derived from a natural host vector for Zika virus do not exhibit a CPE upon Zika infection and do not lyse. The media does not change color and the viability of the C6/36 cells is not altered.

The assay is thus split in two parts: The first part of the assay allows for parallel amplification of potentially live viral particles on 96-well plates of the two susceptible cell lines for six days. The second step of the assay involves the transfer of the supernatant of the 96-well plates (including potentially amplified particles) onto 6-well plates containing monolayers of Vero cells, and incubation for another 8 days to allow for viral infection and a cytopathic effect to develop on the Vero cells. Any CPE observed was confirmed using a light microscope.

Although described in detail with respect to the use of 96 well plates in the first part of the assay, i.e. the culture in C6/36 cells, and six well plates in the second part of the assay, i.e. the culture of Vero cells to observe a cytophatic effect, the assay can be easily scaled up as shown in Table 5 below.

TABLE 5
Details of scale up of Vero cells assay to observe a cytophatic effect

| | | | | | | | | Assay part 2: transfer to | |
|---|---|---|---|---|---|---|---|---|---|
| | | | | | | | | Vero (must accommo- | |
| | | | Assay part 1: | | | | | date pooled volume for | |
| | | | BDS application (must fall within recommended vol range) | | | | | transfer) | |
| | Sur- | Recom- | | vol inocu- | # vessels | # vessels | pooled | mL | vol trans- |
| | face | mended | mL | lum per | required for | required for | volume for | sample | ferred |
| plate or | area | volume range | sample | well (or per | 15× scale-up; | 15× scale-up; | transfer | per | inoculum per |
| flask | (cm²) | (for growth) | per cm² | flask) | 2-fold dilution | 5-fold dilution | (mL) | cm² | well (or flask) |
| 96-well | 0.32 | 100-200 uL | 0.3125 | 0.1 | | | | | |
| format | | | | | | | | | |
| 12-well | 3.8 | 0.076-1.14 ml | 0.3125 | 1.188 | 6.48 | 16.21 | 11.88 | | |
| format | | | | | | | | | |
| 6-well | 9.5 | 1.9-2.9 mL | 0.3125 | 2.969 | 4.32 | 10.81 | 17.81 | 0.0526 | 0.1 |
| format | | | | | | | | | |
| T25 flask | 25 | 5-7.5 mL | 0.3125 | 7.813 | 9.86 | 24.64 | 7.813 | 0.0526 | 1.32 |
| format | | | | | | | | | |
| T75 flask | 75 | 15-22.5 mL | 0.3125 | 23.438 | 3.29 | 8.21 | 23.438 | 0.0526 | 3.95 |
| format | | | | | | | | | |
| T150 flask | 150 | 30-45 mL | 0.3125 | 46.875 | 1.64 | 4.11 | 46.88 | 0.0526 | 7.89 |
| format | | | | | | | | | |
| T175 flask | 175 | 35-52.5 | 0.3125 | 54.688 | 1.41 | 3.52 | 54.69 | 0.0526 | 9.21 |
| format | | | | | | | | | |
| T235 flask | 235 | 47-70.5 | 0.3125 | 73.438 | 1.05 | 2.62 | 73.44 | 0.0526 | 12.36 |
| format | | | | | | | | | |
| T300 flask | 300 | 30-40 mL | 0.3125 | 93.750 | 0.82 | 2.05 | 93.75 | 0.0526 | 15.78 |
| format | | | | | | | | | |
| CF1 | 6/36 | 150-200 | 0.3125 | 198.750 | 0.39 | | | 0.0526 | 33.45 |
| CF2 | 1272 | 300-400 | 0.3125 | 397.500 | 0.19 | | | 0.0526 | 66.91 |
| CF10 | 63360 | 1500-2000 | 0.3125 | 19800.000 | 0.00 | | | 0.0526 | 3332.74 |

It is apparent that during the scale up the volume of sample per cm² of vessel remains constant for part 1 and the same viral infection conditions are kept in part 2.

COI assay control: The titer and back titration controls for this assay were performed using Vero indicator cells and scored in a TCID50 96-well format with wells scored positive based on the media color change from pink to yellow, as a surrogate for cell death, or the presence of CPE.

Virus titer control test: Two independent replicates of the control virus (PRVABC59) of known titer were subjected to a 10-fold dilution series in media containing 2% FBS, and 100 μL of each dilution was added to four wells of a 96-well plate containing Vero cells. Plates were incubated for 5 days, then wells containing CPE were recorded and virus titer was calculated using the Reed-Meunch calculator.

Virus back titration control test: The control virus of known titer was serially diluted to 200 TCID50. Two independent replicates of the 200 TCID50 control virus were subjected to a 2-fold dilution series in media containing 2% FBS, and 100 μL of each dilution was added to four wells of a 96-well plate containing Vero cells. Cells were incubated for 5 days, then wells containing CPE were recorded and virus titer was calculated using the Reed-Meunch calculator.

Detailed COI Protocol:
1. First part of the assay: Vero (1.4E+05 cells/mL) and Aedes aegypti mosquito C6/36 (4E+05 cells/mL) cells were seeded in 96-well plates two days prior to addition of the samples. The Vero cells were cultured in DMEM+10% final FBS+2% L-glutamine+1% penicillin/streptomycin at 37° C. C6/36 cells were cultured in DMEM+10% FBS+2% L-glutamine+1% Penicillin/streptomycin+1% nonessential amino acids at 28° C.
2. Three independent replicates of the 200 TCID50 control virus (prepared in the virus back titration control test) or the DS samples were diluted (5-fold and 10-fold dilutions) into media containing 2% FBS.
3. The cells in 96-well plates were inoculated with the samples. Prior to the infection of the cell monolayers in the 96-well plates, the sample was vortexed to disrupt any possible aggregation. 100 μL of each dilution was applied to each of 5 wells into two separate 96-well plates containing Vero and C6/36 cells, respectively.
4. Media alone was included in another well for each cell type as a negative CPE control.
5. Plates were incubated for 6 days at the appropriate temperature for the cell line.
6. Second part of the assay: To allow live virus to be further amplified and visualized by CPE on a permissive cell line, the entire volume of each 96-well supernatant from both Vero and C6/36 cells was transferred to individual wells of 6-well plates of Vero cells. Inoculation proceeded for 90 minutes with rocking at 15 minutes intervals.
7. Medium containing 2% FBS was added to the wells and plates were incubated for an additional 8 days for subsequent detection of the amplified samples as a function of CPE. The inactivation was considered to be incomplete if any of the replicates of the DS showed CPE at the end of day 8.
7. The presence of live/replicating virions was visualized by the formation of plaques or CPE on susceptible cell monolayers after transfer to the 6-well plate, and incubation for 8 days to allow for viral replication. The % CPE scoring in the 6-well plates at the end of the assay was calculated as follows:
   Each 6-well plate of Vero cells was examined for CPE by visualization of colorimetric change, followed by confirmation of CPE by inspection under an inverted light microscope.
   Each 6-well plate represented one of the replicates of the DS dilutions prepared in the 5 and 10-fold dilutions described above (5 wells, plus one well containing media alone).
   Therefore, % CPE for each replicate reflected the number of wells with CPE out of 5 total wells per sample (120 total wells are used per assay). Mean % CPE and standard deviation were calculated based on three replicates of each dilution.

Results: The daily samples were analyzed in each of the Tox lots #1-4 as shown Tables 6 to 9 below.

TABLE 6
Kinetics of inactivation, Tox lot #1

| | | Mean | |
|---|---|---|---|
| Sample | Transfer | % CPE | STDV |
| 1:10 Day-1 | Vero-to-Vero | 100 | 0 |
| 1:10 Day 0 | Vero-to-Vero | 100 | 0 |
| 1:10 Day 1 | Vero-to-Vero | 0 | 0 |
| 1:10 Day 2 | Vero-to-Vero | 0 | 0 |
| 1:10 Day 3 | Vero-to-Vero | 0 | 0 |
| 1:10 Day 4 | Vero-to-Vero | 0 | 0 |
| 1:10 Day 7 | Vero-to-Vero | 0 | 0 |
| 1:10 Day 8 | Vero-to-Vero | 0 | 0 |
| 1:10 Day 9 | Vero-to-Vero | 0 | 0 |
| 1:10 Day 10 | Vero-to-Vero | 0 | 0 |
| 100TCID50/mL | Vero-to-Vero | 100 | 0 |
| 1:10 Day-1 | C6/36-to-Vero | 100 | 0 |
| 1:10 Day 0 | C6/36-to-Vero | 100 | 0 |
| 1:10 Day 1 | C6/36-to-Vero | 6.7 | 12 |
| 1:10 Day 2 | C6/36-to-Vero | 13.3 | 12 |
| 1:10 Day 3 | C6/36-to-Vero | 0 | 0 |
| 1:10 Day 4 | C6/36-to-Vero | 0 | 0 |
| 1:10 Day 7 | C6/36-to-Vero | 0 | 0 |
| 1:10 Day 8 | C6/36-to-Vero | 0 | 0 |
| 1:10 Day 9 | C6/36-to-Vero | 0 | 0 |
| 1:10 Day 10 | C6/36-to-Vero | 0 | 0 |
| 100TCID50/mL | C6/36-to-Vero | 100 | 0 |

TABLE 7
Kinetics of Inactivation, Tox lot #2

| | | Mean | |
|---|---|---|---|
| Sample | Transfer | % CPE | STDV |
| 1:10 Day-1 | Vero-to-Vero | 100 | 0 |
| 1:10 Day 0 | Vero-to-Vero | 100 | 0 |
| 1:10 Day 1 | Vero-to-Vero | 100 | 0 |
| 1:10 Day 2 | Vero-to-Vero | 0 | 0 |
| 1:10 Day 3 | Vero-to-Vero | 0 | 0 |
| 1:10 Day 4 | Vero-to-Vero | 0 | 0 |
| 1:10 Day 7 | Vero-to-Vero | 0 | 0 |
| 1:10 Day 8 | Vero-to-Vero | 0 | 0 |
| 1:10 Day 9 | Vero-to-Vero | 0 | 0 |
| 1:10 Day 10 | Vero-to-Vero | 0 | 0 |
| 100TCID50/mL | Vero-to-Vero | 100 | 0 |
| 1:10 Day-1 | C6/36-to-Vero | 100 | 0 |
| 1:10 Day 0 | C6/36-to-Vero | 100 | 0 |
| 1:10 Day 1 | C6/36-to-Vero | 100 | 12 |
| 1:10 Day 2 | C6/36-to-Vero | 13.3 | 12 |
| 1:10 Day 3 | C6/36-to-Vero | 0 | 0 |
| 1:10 Day 4 | C6/36-to-Vero | 0 | 0 |
| 1:10 Day 7 | C6/36-to-Vero | 0 | 0 |
| 1:10 Day 8 | C6/36-to-Vero | 0 | 0 |
| 1:10 Day 9 | C6/36-to-Vero | 0 | 0 |
| 1:10 Day 10 | C6/36-to-Vero | 0 | 0 |
| 100TCID50/mL | C6/36-to-Vero | 100 | 0 |

TABLE 8
Kinetics of Inactivation, Tox lot #3

| | | Mean | |
|---|---|---|---|
| Sample | Transfer | % CPE | STDV |
| 1:10 Day-1 | Vero-to-Vero | 100 | 0 |
| 1:10 Day 0 | Vero-to-Vero | 100 | 0 |
| 1:10 Day 1 | Vero-to-Vero | 27 | 12 |
| 1:10 Day 2 | Vero-to-Vero | 0 | 0 |
| 1:10 Day 3 | Vero-to-Vero | 0 | 0 |
| 1:10 Day 4 | Vero-to-Vero | 0 | 0 |
| 1:10 Day 7 | Vero-to-Vero | 0 | 0 |
| 1:10 Day 8 | Vero-to-Vero | 0 | 0 |
| 1:10 Day 9 | Vero-to-Vero | 0 | 0 |
| 1:10 Day 10 | Vero-to-Vero | 0 | 0 |
| 100TCID50/ML | Vero-to-Vero | 100 | 0 |
| 1:10 Day-1 | C6/36-to-Vero | 100 | 0 |
| 1:10 Day 0 | C6/36-to-Vero | 100 | 0 |
| 1:10 Day 1 | C6/36-to-Vero | 87 | 12 |
| 1:10 Day 2 | C6/36-to-Vero | 27 | 12 |
| 1:10 Day 3 | C6/36-to-Vero | 0 | 0 |
| 1:10 Day 4 | C6/36-to-Vero | 0 | 0 |
| 1:10 Day 7 | C6/36-to-Vero | 0 | 0 |
| 1:10 Day 8 | C6/36-to-Vero | 0 | 0 |
| 1:10 Day 9 | C6/36-to-Vero | 0 | 0 |
| 1:10 Day 10 | C6/36-to-Vero | 0 | 0 |
| 100TCID50/mL | C6/36-to-Vero | 100 | 0 |

TABLE 9
Kinetics of Inactivation, Tox lot #4

| | | Mean | |
|---|---|---|---|
| Sample | Transfer | % CPE | STDV |
| 1:10 Day-1 | Vero-to-Vero | 100 | 0 |
| 1:10 Day 0 | Vero-to-Vero | 93 | 12 |
| 1:10 Day 1 | Vero-to-Vero | | 0 |
| 1:10 Day 2 | Vero-to-Vero | 0 | 0 |
| 1:10 Day 3 | Vero-to-Vero | 0 | 0 |
| 1:10 Day 4 | Vero-to-Vero | 0 | 0 |
| 1:10 Day 7 | Vero-to-Vero | 0 | 0 |
| 1:10 Day 8 | Vero-to-Vero | 0 | 0 |
| 1:10 Day 9 | Vero-to-Vero | 0 | 0 |
| 1:10 Day 10 | Vero-to-Vero | 0 | 0 |
| 100TCID50/ML | Vero-to-Vero | 100 | 0 |
| 1:10 Day-1 | C6/36-to-Vero | 100 | 0 |
| 1:10 Day 0 | C6/36-to-Vero | 100 | 0 |
| 1:10 Day 1 | C6/36-to-Vero | 33 | 23 |
| 1:10 Day 2 | C6/36-to-Vero | 7 | 12 |
| 1:10 Day 3 | C6/36-to- Vero | 0 | 0 |
| 1:10 Day 4 | C6/36-to-Vero | 0 | 0 |
| 1:10 Day 7 | C6/36-to-Vero | 0 | 0 |
| 1:10 Day 8 | C6/36-to-Vero | 0 | 0 |
| 1:10 Day 9 | C6/36-to- Vero | 0 | 0 |
| 1:10 Day 10 | C6/36-to-Vero | 0 | 0 |
| 100TCID50/mL | C6/36-to-Vero | 100 | 0 |

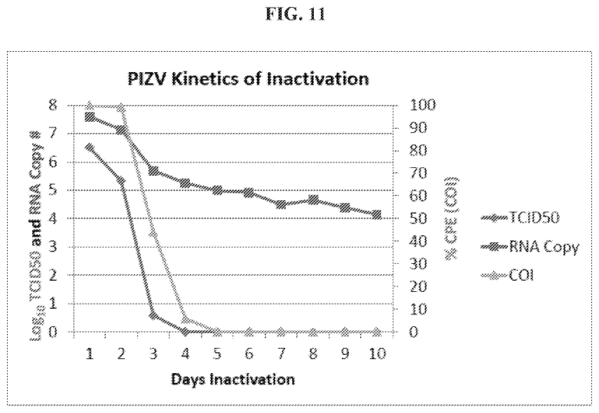

Compiled kinetics of inactivation data: COI data for samples from the four toxicology lots were compared to infectious potency (TCID50) determined as described above and to RNA copy. The RNA copy was determined by purifying nucleic acids from the sample and amplifying Zika RNA with serotype-specific primers using an RT-PCR kit. The result shown in FIG. 11 shows that the sensitivity of the COT assay is significantly greater than that of TCID50.

Performance characteristics of the COI assay—Accuracy: The target dilutions (TCID50/well) and their respective proportions of CPE were used to determine relative accuracy. For the Vero cells, there was a statistically significant linear relationship between the observed and expected proportions of positive CPE. The slope of the line relating observed and expected results is 0.92 with a 95% confidence interval (CI) of 0.83 to 1.01 that overlaps 1 indicates 100% accuracy. For the C6/36 cells, there is a statistically significant linear relationship between the observed and expected proportions of positive CPE. The slope of the line relating observed and expected results is 0.88 with a 95% confidence interval (CI) of 0.80 to 0.95 indicate that a slight bias (5-20%) was seen with this cell line. Both cell lines demonstrate satisfactory accuracy (relative).

Performance characteristics of the COI assay—Limit of Detection (LoD): The sensitivity of the assay was assessed for both the C6/36-to-Vero and Vero-to-Vero plates. As described above, the data was fitted using least squares regression of the proportion of +ve CPE observed per total wells plated with titer dilutions plated starting at 10.00 TCID50/well down to a lower titer of 0.08 TCID50. Furthermore, negative controls (0.00 TCID50/well) were included for each dilution within the plates. CPE scoring was performed for each dilution across both the C6/36-to-Vero and Vero-to-Vero plates. A clear relationship between the CPE and log input virus titer was seen. This displays the logistic (sigmoidal) relationship between the proportion of CPE positive wells relative to the log₁₀ concentration of TCID50/well together with a lower and upper 99% confidence limit. At a −2 log₁₀ concentration (=0.01 TCID50/well), a model based on and accounting for all fixed and random sources variation in the qualification data predicted 0.85%, or 0.01 when rounded up at 0.01 TCID50/well, with a lower 99% confidence limit of 0.42%. Since the lower 99% confidence limit does not include zero, there is a very small quantifiable (<1%) chance the 0.85% CPE wells could have arisen from 0 TCID50/well (i.e., due to noise). This establishes a detection limit for the assay of at least 0.01 TCID50/well (i.e., the lowest amount of live Zika particles in the sample, which can be detected). That is, when rounded up, 1 in 60 wells will be CPE positive or given these parameters, the lowest theoretical proportion of the CPE+ve that could be detected in 60 wells would be 1.67%, or 0.0167.

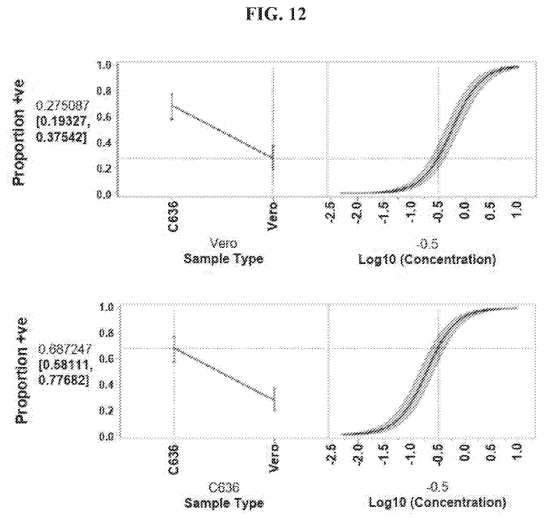

The cell types (C363 and Vero) were compared for relative sensitivity, with the C6/36 demonstrating that a lower dilution of virus titer could be detected compared to Vero cells as shown in FIG. 12; at the same virus input level (0.31 TCID50), the proportion of CPE positive wells is higher for C6/36 relative to Vero cells.

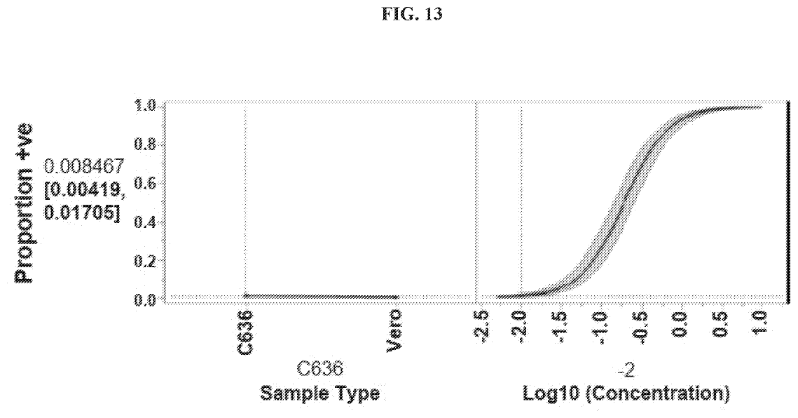

The lowest virus input value used during the qualification of this assay was 0.02 TCID50 (−1.61 log TCID50). Using the fitted curve for C6/36 cells, this results in 0.035 or 3.5% of the wells scoring CPE positive (1 in 28 wells). If the curve is extrapolated towards the lowest practical level of 0.167 or 1.6%, then this equates to a virus input level of 0.015 TCID50 (−1.82 log TCID50). However, the impact of transmitted assay variance needs to be considered when determining the lowest levels of infectious virus that can be detected as reflected in the +ve CPE results. This noise arises from generation of the working stock of input virus. Comparison of the target TCID50 and the back-titration calculation shows the TCID50 of the working stock virus exhibited a standard deviation (SD) of 85 TCID50/mL, derived from a mean of 213 when targeting a stock TCID50/mL concentration of 200. The % CV calculates to ˜40% with a bias of about +7%. This noise was factored into the logistic regression model to generate confidence intervals around the targeted values for the virus dilutions. At a target value of 0.01 TCID50/well, a model based on and accounting for all fixed and random sources of variation in the qualification data across the two sites predicts 0.86% of wells will be CPE positive (1 in 60 wells). Since the lower 99% confidence limit does not include zero, there is a very small quantifiable (<1%) chance the 0.85% CPE-positive wells could have arisen from 0 TCID50/well due to noise (FIG. 13). This establishes a detection limit for the assay: 0.01 TCID50/well is the lowest amount of live Zika particles in the sample which can be detected.

Performance characteristics of the COI assay—Range: The range of the assay was 0.01 TCID50/well to 4.5 TCID50/well and is defined as the range of input virus that resulted in a CPE+ve proportion scoring of more than 0% but less than 100%.

Conclusion: Analysis of the four Tox lots revealed that inactivation was complete after incubation in 0.01% formaldehyde for 10 days at room temperature. Inactivation was achieved by days 3-4 in all lots produced, as measured by the COI assay. The COI assay is more sensitive than TCID50 potency or RNA measurements; the increased sensitivity has also been observed by LoD.

### Example 3: Formulation of the Inactivated Virus Composition

In example 3 the term “Zika virus vaccine drug substance” is used to refer to an inactivated virus composition, which is an intermediate in the production of a Zika vaccine.

### Equipment

TABLE 10
Materials used in Example 3

| | |
|---|---|
| Materials | Manufacturer/Supplier |

Materials for preparation of the drug substance
Buffer salts and additives

| | |
|---|---|
| Disodium phosphate | EMPRove bio 10028-24-7 |
| Dipotassium phosphate | Fisher P290-500 |
| Tris(hydroxymethyl)aminomethane | Fisher T395-500 |
| base | |
| Histidine | J.T. Baker N327-05 |
| Sodium Chloride | Fisher S271-500 |
| Sucrose | Fisher S3-212 |
| Hydrochloric Acid | Fisher A1445-500 |
| Sodium Hydroxide Solution | Fisher SS255-1 |

Materials used in aliquotino the samples

| | |
|---|---|
| 3 mL vials | Nuova Ompi, Italy, Ez-Fill, Nest & tub, ISO- |
| | 2R vials, no siliconization, ready to use (pre- |
| | sterilized), |
| ETFE laminated stopper | Daikyo Seiko, Japan, Item: S2-F45-2, |
| | Formulation: D777-1, ready to use stopper |
| | (pre-sterilized) |

Materials used in size exclusion chromatography (SEC)

| | |
|---|---|
| 2 mg/mL BSA solution | Thermo/Pierce Catalog #23209 |
| Sterile syringe filters (0.22 μm Millex | EMD Millipore |
| GV) | |
| HPLC vials | Agilent #5188-6591 Low-volume(fixed |
| | insert) vials |
| HPLC vial caps | National Scientific Catalog #C4000-54B |

Where water is mentioned in the experimental section MilliQ water, with a resistivity of 18 MΩ is meant.

The buffers used and their abbreviations are listed in Table 11.

TABLE 11
Buffers used in Example 3

| Buffer | Abbreviation | pH |
|---|---|---|
| Zika Phosphate Buffer | ZPB | 7.4 |
| 6.46 mM Disodium phosphate | | |
| 1.47 mM Dipotassium phosphate | | |
| 137 mM NaCl | | |
| Tris Buffer¹ | Tris | 7.6 |
| 10 mM Tris(hydroxymethyl)aminomethane base | | |
| 20 mM NaCl | | |
| Histadine Buffer¹ | His | 7.0 |
| 20 mM Histadine | | |
| 20 mM NaCl | | |
| Tris buffered saline | TBS | 7.6 |
| 50 mM Tris(hydroxymethyl)aminomethane base | | |
| 150 mM NaCl | | |

¹Where not otherwise specified Tris + Sucrose and His + Sucrose refers to a 7% w/v sucrose solution in Tris or Histidine buffer.

Buffers were all titrated to the correct pH with HCl or NaOH, as needed.

### Manufacture of the Zika Vaccine Drug Substance

Purified Inactivated Zika Vaccine drug substance was manufactured by growth in Vero cells as described above. Daily harvests were carried out on days 3 to 9, and these were pooled prior to purification and inactivation. Supernatants from the daily harvests were purified by filtration and chromatography, concentrated and inactivated by addition of formaldehyde to a final concentration of 0.01%. Inactivation was allowed to proceed for 10 days at 22° C., before the sample was neutralized with sodium metabisulfite and then buffer exchanged into Zika Phosphate Buffer (6.46 mM disodium phosphate, 1.47 mM dipotassium phosphate, 137 mM NaCl, 6% sucrose, pH 7.4).

### Buffer Exchange

Once manufactured, the Zika virus vaccine drug substance was stored at +5±3° C./ambient humidity for up to 6 months.

The Zika virus vaccine drug substance was then buffer exchanged into the appropriate buffers used in each of the examples (as listed in table 11) using tangential flow filtration (TFF), as described below. Tangential flow filtration was carried out using the equipment listed in Table 12: The buffer exchange process was carried out at room temperature ca. 25° C.

TABLE 12
Materials and equipment for the buffer exchange procedure

| Equipment | Manufacturer/Supplier |
|---|---|
| TFF system | Spectrum Labs KR2i |
| Main pump | Spectrum Labs 900-1893 |
| Auxiliary pump | Spectrum Labs 708-13683-000 |
| Balances (permeate and feed) | Spectrum Labs |
| Automatic back pressure control | Spectrum Labs |
| valve | |
| KR2i data collection software | Spectrum Labs |
| Main pump | Spectrum Labs 900-1893 |
| MicroKros hollow fiber filter | Spectrum Labs C02-E100-05-N |
| module (mPES/100 kD) | |
| Process reservoir conical bottom, | Spectrum Labs ACBT-015-C1N |
| 15 ml | |
| Pharmapure tubing pack size 13 | Spectrum Labs ACTU-P13-25N |
| Pressure transducer | Spectrum Labs ACPM-799-01N |
| Fittings kit | Spectrum Labs |

Tangential flow filtration (TFF) was carried out using the following procedure:

Setup: the KR2i TFF system was set up according to the manufacturer's instructions (KR2i/KMPi TFF Systems product information and operating instructions 2016, http://spectrumlabs.com/lit/400-12355-000rev02.pdf). This involves connecting the TFF column (MicroKros hollow fiber filter module (mPES/100 kD) Spectrum Labs C02-E100-05-N) to the TFF system. The column was then washed with 50 to 150 mL of water 10 and subsequently equilibrated with approximately 100 mL of the appropriate buffer. During the washing and equilibrium steps, the flow rate was maintained at around 25 mUmin and the total pressure was not allowed to exceed 18 psi. at any time.

Concentration: initially, 5 to 40 mL of sample (Zika virus vaccine drug substance) were added to the sample reservoir and was concentrated by a factor of 2.

Diafiltration: after concentration, diafiltration was performed. Diafiltration is the act of diluting and filtering a sample of Zika virus vaccine drug substance at the same time. Dilution adds volume to the sample, whereas filtration decreases the volume of the sample.

Diafiltration was carried out by starting the auxiliary pump (for diafiltration) and using a flow rate sufficient to maintain the sample volume during diafiltration i.e. ˜0.5-1.5 mL per min. The pressure was manually controlled throughout, using a backpressure control valve. The pressure was maintained between 14-18 psi., the sheer used was no more than 5000, the flux was between 66-72, the flow rate (main pump) was between 25-28 mL/min and the auxiliary flow rate (auxiliary pump) was between 0.5 and 1 mL/min. The auxiliary pump controls the rate of dilution. This rate is manually adjusted to match the filtration rate (so that the net sample volume does not change during this step). The filtration rate is complex, as it is determined by many factors (flow rate of the main pump, transmembrane pressure, degree of column fowling, etc.) and the rate changes throughout the run; consequently, the volume of the sample is controlled by modulating the auxiliary pump flow rate manually.

In order to ensure complete diafiltration, at least 10 diavolumes of the appropriate (new) buffer were exchanged (as measured by the accumulated mass of the permeate). In the current example, constant-volume diafiltration (continuous diafiltration) was performed. This involves keeping the volume of the drug substance (retentate) during the filtration process constant, by adding the new buffer at the same rate as the filtrate is removed. The number of diavolumes exchange can be calculated using the following equation:

$$Number of diavolumes = \frac{ \begin{matrix}Total volume of new buffer introduced \\ during the diafiltration procedure\end{matrix} }{Volume of the drug substance ( retentate )}$$

wherein, the volume of drug substance (retentate) remains constant throughout the process.

The diafiltration process provided samples of Zika virus vaccine drug substance (DS), which have been buffer exchanged into the buffers listed in table 11.

Sample recovery and storage: on some occasions, the sample was further concentrated following diafiltration (up to approximately 10 times). This was carried out by closing the auxiliary pump and continuing to filter (i.e. leaving the main pump switched on). In order to recover the sample at the end of the diafiltration process the feed and recirculation lines were raised above the level of the sample and the flow of the pump was reversed to collect the residual volume (generally around 1-3 mL). Appropriate confirmatory assays such as pH determination, osmolality, and SEC were subsequently performed for the final product (the buffer exchanged Zika virus vaccine drug substance) after TFF.

The Zika virus vaccine drug substance (DS) after buffer exchange was then stored at +5±3° C./ambient humidity for up to 5 days before carrying out the stability tests.

### Stability Test Procedure

At the beginning of each study (examples 3A-3F), samples of Zika virus vaccine drug substance (DS) following buffer exchange were aliquoted into individual 3 mL vials, with each vial containing approximately 0.67 mL of the Zika virus vaccine drug substance (DS) in the respective buffer. The vials were then sealed with an ETFE laminated stopper.

A single sample, corresponding to one aliquot, for each different buffer was then tested immediately using size exclusion chromatography (SEC, as described below) to determine the peak area (and, therefore, the amount of protein in μg/mL) at the beginning of the study (day 0/freeze-thaw cycle 0).

Sufficient vials of Zika virus vaccine drug substance (DS) in each of the respective buffers were placed at +5±3° C., ambient humidity and −80° C., ambient humidity at the beginning of each study. For each time point and temperature condition measured, a separate vial was prepared.

Samples stored at −80° C. were frozen in a −80° C. chamber. Due to the 0.67 mL fill volume, the samples froze relatively rapidly but were not “snap frozen” in liquid nitrogen.

### Size Exclusion Chromatography (SEC) Procedure

Analysis of stability was performed using size exclusion chromatography (SEC) using a single column. Size exclusion chromatography is a technique used to separate proteins based on their molecular weight. The larger the molecular weight of a protein sample, the shorter its elution time. A peak in the SEC trace was seen with a retention time of around 8 minutes for the intact Zika virus. By comparing the integral of this peak with the integral of a reference sample (at day zero) it is possible to determine how much intact Zika virus is still present in the sample after a period of storage. Furthermore, by noting whether any further peaks appear at earlier retention times, it is possible to determine whether the Zika virus has agglomerated.

The equipment and materials used for size exclusion chromatography (SEC) are detailed in Table 13 below.

TABLE 13
Equipment used for SEC

| Equipment | Specification |
|---|---|
| HPLC (High Performance | Agilent 1100 or 1260 |
| Liquid | |
| Chromatography) System | |
| Data System Method | LP005.M |
| Reprocessing Method | LP005R.M |
| Column | Superose-6 Increase (GE Healthcare) |
| Mobile Phase | 1x PBS (10.14 mM Sodium Phosphate, |
| | Dibasic; 1.79 mM Potassium Phosphate, |
| | Monobasic:, 136.89 mM NaCl, 2.68 |
| | mM KCl, 250 ppm NaN₃ pH 7.4) |
| Flow rate (mL/min) | 1.0 |
| UV-VIS Detector | 280 nm |
| Wavelengths | |
| Column Temperature | RT (not regulated) |
| Auto-Sampler Tray | 5° C. |
| Temperature | |

Size exclusion chromatography (SEC) was carried out using the procedure detailed below.

### Preparation of Standards and Test Samples

BSA reference standard: reference samples of bovine serum albumin (BSA) with concentrations of 200, 400, 800, and 1,000 μg/mL were prepared by diluting the BSA stock solution, which has a well-defined concentration of 2 mg/mL, with water. An aliquot of each reference sample was then transferred into a labeled HPLC vial and capped. All protein concentrations given in examples 3A-3F below are based on the concentration of Zika virus based on a BSA standard curve.

Preparation of samples for SEC: At each specific time point tested, the required 3 mL vials were removed from storage at +5±3° C. or −80° C. Samples frozen at −80° C. were thawed at room temperature. SEC samples were then prepared by transferring at least 400 μL of each test sample from the appropriate 3 mL vial into a labeled HPLC vial and capping the vial. The HPLC vials were then placed into the HPLC auto-sampler tray. The HPLC auto-sampler tray was cooled at 5 f 3° C., ensuring that all of the SEC samples were cooled to 5±3° C. before SEC was carried out.

Measurement: Each SEC measurement involved injecting a 100 μL aliquot of each of the samples into the SEC column using an auto sampler/auto injector.

### Calculation Used to Determine the Concentration of Zika Protein from the SEC Chromatograms

For each SEC run, a standard curve of bovine serum albumin (BSA), was produced, standards of 200, 400, 800, and 1,000 μg/mL concentration were run and a standard curve was prepared (linear regression, y=mx+b with b=0). The BSA total peak area was calculated as the sum of the monomer and dimer peak areas for BSA.

This standard curve was used to determine the concentration of Zika virus vaccine drug substance (DS) (in terms of concentration of BSA in μg/mL).

The values for concentration were then normalized relative to the measurement taken on day 0 for each of the samples (which was taken as the 100% value).

The Zika virus vaccine drug substance (DS) peak areas were determined as the entire peak eluting after a retention time of approximately 8 minutes in the SEC chromatograms. Peak fitting was performed by connecting the baseline before and after the peak at around 8 minutes. This includes the main peak and any further shoulder that elutes with a shorter retention time (i.e. to the left of the peak). Peaks or shoulders with a significantly longer retention time were not included in the integration, as these peaks are likely to correspond to degraded or denatured Zika virus proteins.

Concentration determination of Zika virus vaccine drug substance (DS): The total mass of the Zika virus vaccine drug substance (DS) was calculated based on the BSA calibration curve. Zika virus vaccine drug substance (DS) concentration is reported in μg/mL and is calculated by dividing the mass obtained from the calibration curve by 0.1 mL (see Equation 1).

$$Zika DS Concentration = [ \frac{Mass of Zika DS ( µg )}{0.1 mL} ] \qquad Equation 1$$

The results given for each of the examples are an average of two repeat SEC readings for the same sample.

### Example 3A

Preparation of the samples in example 3A was carried out as described above. Zika virus vaccine drug substance was prepared and then buffer exchanged into each of the respective buffers listed in tables 14a and 14b below. In example 3A, stability tests were carried out over a 10 day period at both 5±3° C. and −80° C.

Tables 14a and 14b show the results of SEC carried out after 0 and 10 days, for Zika virus vaccine drug substance samples in a number of different buffers. The results of these experiments are given as percentages, based on the area of the SEC peak at day 10 as a percentage of the area of the peak at day 0.

TABLE 14a
SEC results for Example 3A at 5 ± 3° C.

| | Peak area/normalized to the BSA | | Percentage |
|---|---|---|---|
| | concentration standard [μg/mL] | | remaining at |
| | Day 0 | Day 10 | day 10/% |
| Buffer | | | |
| ZPB | 133 | 112 | 85 |
| Tris | 133 | 104 | 78 |
| Tris + 7% Sucrose | 135 | 111 | 83 |

TABLE 14b
SEC results for Example 3A at −80° C.

| | Peak area/normalized to the BSA | | Percentage |
|---|---|---|---|
| | concentration standard [μg/mL] | | remaining at |
| | Day 0 | Day 10 | day 10/% |
| Buffer | | | |
| ZPB | 133 | 78 | 58 |
| Tris | 133 | 88 | 66 |
| Tris + 7% Sucrose | 135 | 109 | 81 |

### Example 3B

Preparation of the samples in example 3B was carried out as described above. Zika virus vaccine drug substance was prepared and then buffer exchanged into each of the respective buffers listed in the table below. In example 3B, stability tests were carried out over a 60 day period at −80° C.

Table 15 shows the results of SEC carried out after 0 and 60 days, for Zika virus vaccine drug substance samples in a number of different buffers. The results of these experiments are given as percentages, based on the area of the SEC peak at day 60, as a percentage of the area of the peak at day 0.

TABLE 15
SEC results for Example 3B at −80° C.

| | Peak area/normalized to the BSA | | Percentage |
|---|---|---|---|
| | concentration standard [μg/mL] | | remaining at |
| | Day 0 | Day 60 | day 60/% |
| Buffer | | | |
| His | 27 | 11 | 41 |
| His + 7% Sucrose | 31 | 20 | 64 |

### Example 3C

Preparation of the samples in example 3C was carried out as described above. Zika virus vaccine drug substance was prepared and then buffer exchanged into each of the respective buffers listed in the table below. In example 3C, stability tests were carried out over a 67 day period at both 5 f 3° C. and −80° C.

Tables 16a and 16b show the results of SEC carried out after 0 and 67 days, for Zika virus vaccine drug substance samples in a number of different buffers. The results of these experiments are given as percentages, based on the area of the SEC peak at day 67 as a percentage of the area of the peak at day 0.

TABLE 16a
SEC results for Example 3C at 5 ± 3° C.

| | Peak area/normalized to the BSA | | Percentage |
|---|---|---|---|
| | concentration standard [μg/mL] | | remaining at |
| | Day 0 | Day 67 | day 67/% |
| Buffer | | | |
| ZPB | 95 | 83 | 88 |
| Tris + 7% Sue | 79 | 75 | 95 |
| Tris + 10% v/v | 78 | 81 | 104 |
| Glycerol | | | |

TABLE 16b
SEC results for Example 3C at −80° C.

| | Peak area/normalized to the BSA | | | Percentage | |
|---|---|---|---|---|---|
| | concentration standard [μg/mL] | | | remaining/% | |
| | Day 0 | Day 1 | Day 67 | Day 1 | Day 67 |
| Buffer | | | | | |
| ZPB | 95 | 68 | 65 | 72 | 69 |
| Tris + 7% Suec | 79 | 74 | 62 | 94 | 78 |
| Tris + 10% v/v | 78 | 79 | 81 | 101 | 104 |
| Glycerol | | | | | |

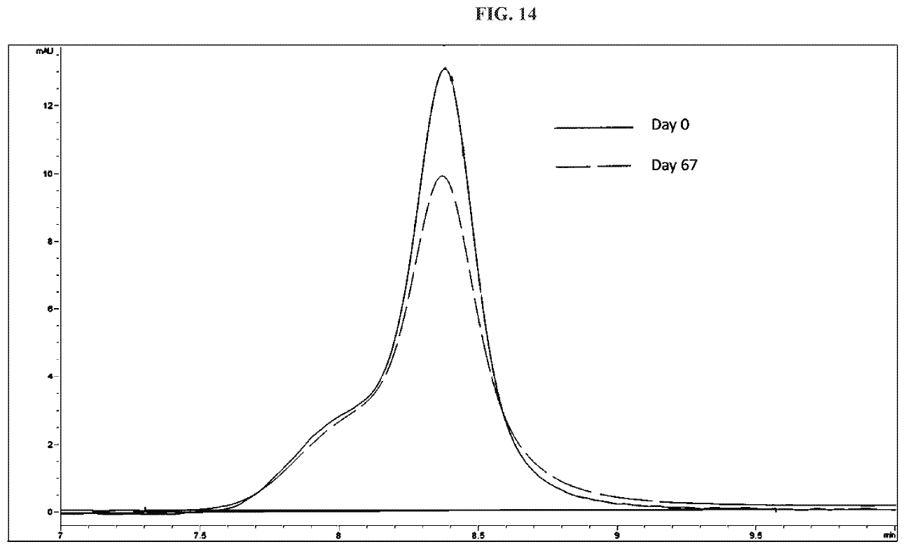

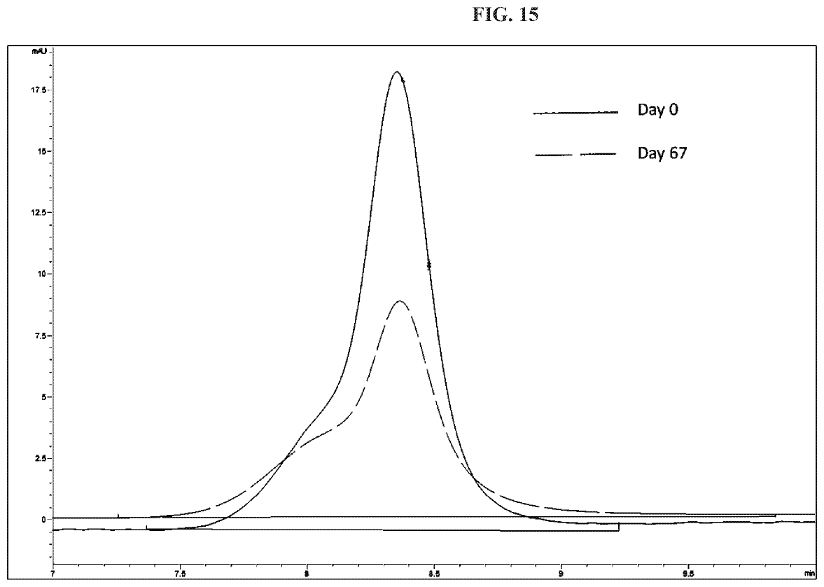

Additionally, FIGS. 14 and 15 show the SEC chromatograms for zika virus vaccine drug substance stored in Tris+7% sucrose buffer and in ZPB buffer at −80° C. 67 days. For drug substance in Tris+7% sucrose buffer (FIG. 14) the peak shape at day 67 is very similar to the peak shape at day 0. In contrast, for the drug substance in ZPB (FIG. 15), at day 67 the chromatographic profile has shifted, in particular the size of the main peak has decreased and the shoulder peak has increased in size (i.e. the shoulder peaks lifts off from the base line at around 7.5 minutes), this suggests aggregation.

### Example 3D

Preparation of the samples in example 3D was carried out as described above. Samples of Zika virus vaccine drug substance were prepared and then buffer exchanged into Tris and ZPB buffer (as listed in the table 11 above). In example 3D, stability tests were carried out over a 3 month period at −80° C.

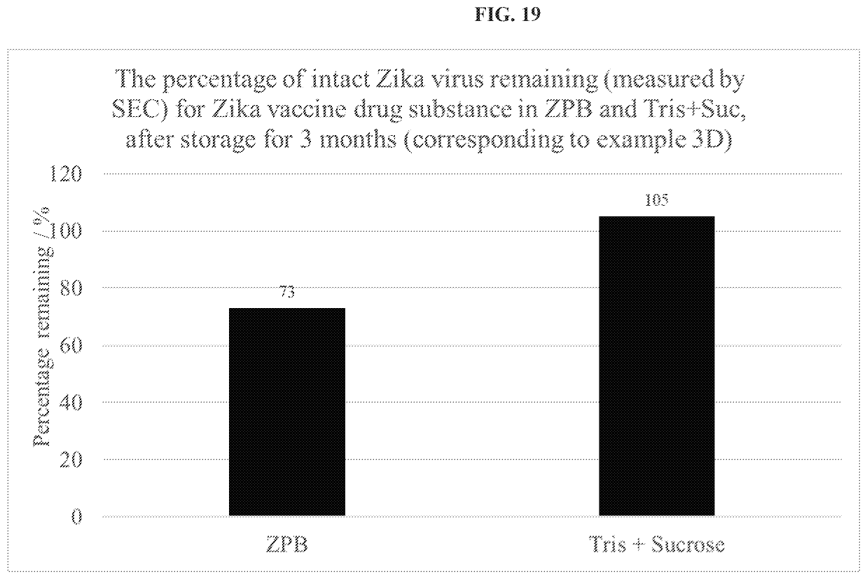

The results for this study are shown in FIG. 19.

### Example 3E

Preparation of the samples in example 3E was carried out as described above. Zika virus vaccine drug substance was prepared and then buffer exchange into each of the respective buffers listed in the table below. In example 3E, stability tests were carried out over a 60 day period at 5 t 3° C.

Table 17 shows the results of SEC carried out after 1, 3, 8, 15, 30 and 60 days, for Zika virus vaccine drug substance samples in ZPB and TBS. The results of these experiments are given as percentages, based on the area of the SEC peak at day 1, 3, 8, 15, 30 and 60 as a percentage of the area of the peak at day 0.

TABLE 17
SEC results for Example 3E at 5 ± 3° C.

| | | Day | | | | | | |
|---|---|---|---|---|---|---|---|---|
| | Buffer | 0 | 1 | 3 | 8 | 15 | 30 | 60 |
| Peak area/content as BSA | ZPB | 102 | 96 | 98 | 84 | 86 | 75 | 69 |
| [μg/mL] | TBS | 99 | 93 | 92 | 87 | 88 | 87 | 83 |
| Percentage remaining/% | ZPB | — | 94 | 96 | 82 | 84 | 73 | 68 |
| | TBS | — | 93 | 92 | 88 | 89 | 88 | 84 |

### Example 3F

Preparation of the samples in example 3F was carried out as described above. Zika virus vaccine drug substance was prepared and then buffer exchanged into Tris buffer. Sucrose was subsequently added to the samples (to achieve samples with a total concentration of 3, 5, 7 and 10% w/v respectively).

Table 18 shows the results of SEC carried out after 0, 1, 2, 3, and 4 freeze thaw cycles.

A tray of samples was prepared in individual vials. Each freeze thaw cycle involved warming the tray (containing all of the samples) to room temperature (25° C.) by defrosting at room temperature. A single vial under each of the conditions was taken for analysis after each freeze thaw cycle.

Results are given as percentages, based on the area of the SEC peak before and after 1 to 4 freeze thaw cycles.

TABLE 18
SEC column results for multiple freeze thaw cycles

| | | Freeze thaw cycle | | | | |
|---|---|---|---|---|---|---|
| | Buffer | 0 | 1 | 2 | 3 | 4 |
| Peak area/content | Tris + 3% sucrose | 288 | 277 | 259 | 252 | 207 |
| as BSA [μg/mL] | Tris + 5% sucrose | 286 | 271 | 254 | 244 | 206 |
| | Tris + 7% sucrose | 284 | 278 | 267 | 268 | 267 |
| | Tris + 10% sucrose | 287 | 280 | 286 | 276 | 272 |
| Percentage | Tris + 3% sucrose | — | 95 | 90 | 88 | 72 |
| remaining/% | Tris + 5% sucrose | — | 95 | 89 | 85 | 72 |
| | Tris + 7% sucrose | — | 98 | 94 | 94 | 94 |
| | Tris + 10% sucrose | — | 98 | 100 | 96 | 95 |

1. A liquid inactivated virus composition comprising:
a) an inactivated whole Zika virus,
b) at least one pharmaceutically acceptable buffer with a concentration of at least about 6.5 mM, and
c) optionally a polyol,
wherein the liquid inactivated virus composition preferably does not contain an adjuvant selected from aluminum salts and
said at least one pharmaceutically acceptable buffer does not comprise phosphate ions.

2. The liquid inactivated virus composition of claim 1, wherein the concentration of phosphate ions in the liquid inactivated virus composition is less than about 7 mM, or less than about 6 mM, or less than about 5 mM, or less than about 4 mM, or less than about 3 mM, or less than about 2 mM, or less than about 1 mM.

3. The liquid inactivated virus composition of claims 1 or 2, wherein the liquid inactivated virus composition comprises only one pharmaceutically acceptable buffer.

4. The liquid inactivated virus composition of claims 1 or 2, wherein the liquid inactivated virus composition comprises at least two different pharmaceutically acceptable buffers, wherein the molar ratio of the two most concentrated pharmaceutically acceptable buffers in the liquid inactivated virus composition is not between 1:2 to 2:1, or not between 1:5 to 5:1, or not between 8:1 to 1:8, or not between 10:1 to 1:10.

5. The liquid inactivated virus composition of any one of the preceeding claims, wherein the concentration of potassium ions in the liquid inactivated virus composition is less than about 4 mM, or less than about 3 mM, or less than about 2 mM, or less than about 1.5 mM, or less than about 0.5 mM, or less than about 0.1 mM, or about 0 mM.

6. The liquid inactivated virus composition of any one of the preceeding claims, wherein the liquid inactivated virus composition is substantially free or free of protamine sulphate.

7. The liquid inactivated virus composition of any one of the preceeding claims, wherein the pH of the liquid inactivated virus composition is from about pH 6.0 to about pH 9.0 or from about pH 6.5 to about pH 8.0, or from about pH 6.8 to about pH 7.8, or about pH 7.6, as determined at room temperature.

8. The liquid inactivated virus composition of any one of the preceeding claims, wherein the concentration of the pharmaceutically acceptable buffer is at least about 7 mM, or at least about 7.5 mM, or at least about 8 mM, or at least about 8.5 mM, or at least about 9 mM, or at least about 10 mM.

9. The liquid inactivated virus composition of claim 8, wherein the concentration of the pharmaceutically acceptable buffer is from about 7 mM to about 200 mM, or from about 7.5 mM to about 200 mM, or from about 8 mM to about 200 mM, or from about 8.5 mM to about 200 mM, or from about 9 mM to about 100 mM, or from about 9 mM to about 60 mM, or about 10 mM, or about 20 mM, or about 50 mM.

10. The liquid inactivated virus composition of any one of the preceeding claims, wherein the pharmaceutically acceptable buffer comprises an amino group-containing molecule.

11. The liquid inactivated virus composition of claim 10, wherein the pharmaceutically acceptable buffer is Tris or Histidine buffer.

12. The liquid inactivated virus composition of claim 11, wherein the pharmaceutically acceptable buffer is Tris.

13. The liquid inactivated virus composition of any one of the preceding claims, wherein the composition further comprises at least one polyol.

14. The liquid inactivated virus composition of claim 13, wherein the liquid inactivated virus composition comprises from about 1% w/v to about 60% w/v of the polyol, or from about 6% w/v to about 50% w/v of the polyol, or from about 6% w/v to about 40% w/v of the polyol, or from about 6% w/v to about 35% w/v of the polyol, or from about 6% w/v to about 30% w/v of the polyol, or from about 6% w/v to about 25% w/v of the polyol, or from about 6% w/v to about 20% w/v of the polyol, or from about 6% w/v to about 15% w/v of the polyol, or from about 6% w/v to about 12% w/v of the polyol, or about 7% w/v of the polyol, or about 10% w/v of the polyol.

15. The liquid inactivated virus composition of claim 14, wherein the liquid inactivated virus composition comprises a pharmaceutically acceptable buffer comprising an amino group-containing molecule, and from about 6% w/v to about 15% w/v of a polyol.

16. The liquid inactivated virus composition of claim 15, wherein the liquid inactivated virus composition comprises Tris, and from about 6% w/v to about 15% w/v of a polyol.

17. The liquid inactivated virus composition of any one of claims 13 to 16, wherein the polyol is a sugar.

18. The liquid inactivated virus composition of claim 17, wherein the sugar is a disaccharide.

19. The liquid inactivated virus composition of claim 18, wherein the disaccharide is a non-reducing sugar.

20. The liquid inactivated virus composition of claim 19, wherein the non-reducing sugar is sucrose.

21. The liquid inactivated virus composition of claim 20, wherein the liquid inactivated virus composition comprises from about 5% w/v to about 20% w/v sucrose, or from about 6% w/v to about 15% w/v sucrose.

22. The liquid inactivated virus composition of claim 21, wherein the liquid inactivated virus composition comprises from about 6% w/v to about 8% w/v sucrose, such as about 7% w/v sucrose.

23. The liquid inactivated virus composition of claim 21, wherein the liquid inactivated virus composition comprises from about 8.5 mM to about 50 mM Tris and from about 6% to about 15% w/v sucrose, wherein the pH of the inactivated virus composition is from about pH 7.0 to about pH 8.0, when measured at room temperature.

24. The liquid inactivated virus composition of claim 13, wherein the polyol is glycerol.

25. The liquid inactivated virus composition of claim 24, wherein the liquid inactivated virus composition comprises from about 1% v/v to about 60% v/v glycerol, or from about 7% v/v to about 15% v/v glycerol, or about 10% v/v of glycerol.

26. The liquid inactivated virus composition of claim 25, wherein the inactivated virus composition comprises from about 8.5 mM to about 50 mM Tris and from about 6% v/v to about 15% v/v glycerol, wherein the pH of the inactivated virus composition is from about pH 7.0 to about pH 8.0, when measured at room temperature.

27. The liquid inactivated virus composition of any one of the preceding claims, wherein the liquid inactivated virus composition further comprises sodium chloride.

28. The liquid inactivated virus composition of claim 27, wherein the concentration of sodium chloride in the liquid inactivated virus composition is from about 5 mM to about 500 mM sodium chloride, or from about 10 mM to about 200 mM sodium chloride.

29. The liquid inactivated virus composition of claim 28, wherein the concentration of sodium chloride in the liquid inactivated virus composition is from about 10 mM to about 40 mM, or from about 10 mM to about 30 mM, such as about 20 mM.

30. The liquid inactivated virus composition of any one of the preceding claims, wherein the ionic strength of the liquid inactivated virus composition is below about 80 mM, or below about 70 mM, or below about 60 mM, or below about 50 mM, or below about 40 mM, or below about 30 mM.

31. The liquid inactivated virus composition of any one of the preceding claims, wherein the Zika virus is an African lineage virus or an Asian lineage virus.

32. The liquid inactivated virus composition of claim 31, wherein the Zika virus is an Asian lineage virus.

33. The liquid inactivated virus composition of claim 32, wherein the Zika virus is derived from strain PRVABC59.

34. The liquid inactivated virus composition of claim 33, wherein strain PRVABC59 comprises the genomic sequence according to SEQ ID NO: 2.

35. The liquid inactivated virus composition of any one of the preceding claims, wherein the Zika virus has a mutation at position 98 of SEQ ID NO: 1, or at a position corresponding to position 98 of SEQ ID NO: 1.

36. The liquid inactivated virus composition of claim 35, wherein the mutation is a Trp98Gly mutation in SEQ ID NO: 1.

37. The liquid inactivated virus composition of any one of the preceding claims, wherein the Zika virus does not comprise a mutation in the envelope protein (Env).

38. The liquid inactivated virus composition of any one of the preceding claims, wherein the sequence encoding the envelope protein is the same as the corresponding sequence in SEQ ID No. 2, or wherein the sequence of the envelope protein shows at least 99% identity, or at least 95% identity, or at least 90% identity, or at least 85% identity with the sequence in SEQ ID No. 2.

39. The liquid inactivated virus composition of any one of the preceding claims, wherein the Zika virus is at least 85% pure as determined by the main peak of the purified Zika virus in the size exclusion chromatography being more than 85% of the total area under the curve in the size exclusion chromatography.

40. The liquid inactivated virus composition of any one of the preceding claims, wherein the Zika virus is chemically inactivated.

41. The liquid inactivated virus composition of claim 40, wherein the Zika virus is inactivated with one or more of a detergent, formaldehyde, hydrogen peroxide, beta-propiolactone (BPL), binary ethylamine (BEI), acetyl ethyleneimine, methylene blue, and psoralen.

42. The liquid inactivated virus composition of claim 41, wherein the Zika virus is inactivated with formaldehyde.

43. The liquid inactivated virus composition of claim 42, wherein the Zika virus is an inactivated whole virus obtainable from a method wherein the Zika virus is treated with formaldehyde in an amount that ranges from about 0.001% w/v to about 3.0% w/v from 5 to 15 days at a temperature that ranges from about 15° C. to about 37° C.

44. The liquid inactivated virus composition of claim 43, wherein the Zika virus is an inactivated whole virus obtainable by treating a whole live Zika virus with 0.005% to 0.02% w/v of formaldehyde.

45. The liquid inactivated virus composition of claim 44, wherein the Zika virus is an inactivated whole virus obtainable by treating a whole live Zika virus with less than 0.015% w/v of formaldehyde.

46. A liquid vaccine comprising:
a) the inactivated virus composition according to any one of the preceding claims, and
b) an adjuvant such as aluminum hydroxide.

47. The liquid vaccine of claim 46, wherein the concentration of sodium chloride in the liquid vaccine is from about 50 mM to about 200 mM, or from about 60 mM to about 150 mM, such as about 84 mM.

48. The liquid vaccine of claim 47, wherein the liquid vaccine comprises from about 8.5 mM to about 80 mM Tris and from about 60 mM to about 150 mM NaCl, and wherein the pH of the liquid inactivated virus composition is from about pH 7.0 to about pH 8.0, when measured at room temperature.

49. The liquid vaccine of claim 48, wherein the liquid vaccine comprises from about 0.4% (w/v) to 4.7% (w/v) sucrose.

50. The liquid vaccine of claim 49, comprising 100 μg/ml to 800 μg/ml aluminum hydroxide, or 200 μg/ml to 600 μg/ml aluminum hydroxide, or 300 μg/ml to 500 μg/ml aluminum hydroxide, or about 400 μg/ml aluminum hydroxide based on elemental aluminum.

51. A unit dose of the liquid vaccine according to any one of claims 46 to 50.

52. The unit dose of vaccine of claim 51 comprising from about 1 μg to about 15 μg of the inactivated whole Zika virus.

53. The unit dose of vaccine of claim 52, comprising about 2 μg of inactivated whole Zika virus.

54. The unit dose of vaccine of claim 52, comprising about 5 μg of inactivated whole Zika virus.

55. The unit dose of vaccine of claim 52, comprising about 10 μg of inactivated whole Zika virus.

56. The unit dose of vaccine of any one of claims 51 to 55 provided as about 0.4 mL to about 0.8 mL of a pharmaceutically acceptable liquid.

57. Use of an inactivated virus composition comprising:
a) an inactivated whole Zika virus,
a) at least one pharmaceutically acceptable buffer with a concentration of at least about 6.5 mM, and
b) optionally a polyol,
wherein the inactivated virus composition does not contain an adjuvant selected from aluminum salts and said at least one pharmaceutically acceptable buffer does not comprise phosphate ions,
for stabilizing the inactivated whole Zika virus.

58. The use of claim 57, for stabilizing the inactivated whole Zika virus during storage at 5±3° C. for at least 10 days.

59. The use of claim 57, for stabilizing the inactivated whole Zika virus during storage at −80° C. for at least 10 days.

60. The use of claim 59, for stabilizing the inactivated whole Zika virus during storage at −80° C. for at least 6 months.

61. The use of claim 60, for stabilizing the inactivated whole Zika virus during storage at −80° C. for at least 12 months.

62. The use of any one of claims 57 to 61, for stabilizing the inactivated whole Zika virus during one or multiple freeze thaw cycles, such as at least 2 freeze thaw cycles, or at least 4 freeze thaw cycles.

63. A method of treating or preventing, in particular preventing Zika virus infection in a human subject, in need thereof, comprising administering to the subject the unit dose of vaccine of any one of claims 51 to 56.

64. The unit dose of vaccine of any one of claims 51 to 56, for use in treating or preventing, in particular preventing a Zika virus infection in a human subject, in need thereof.

65. Use of a unit dose of vaccine according to any one of claims 51 to 56, in the manufacture of a medicament for preventing a Zika virus infection in a human subject, in need thereof.

66. A method of preparing a liquid inactivated virus composition comprising:
a) an inactivated whole Zika virus,
b) a pharmaceutically acceptable buffer, wherein the said buffer is not phosphate buffer and wherein the concentration of said buffer is at least 6.5 mM; and
c) optionally a polyol;
wherein the inactivated virus composition preferably does not contain an adjuvant selected from aluminum salts; the method comprising the following steps:
Step 1. isolating a Zika virus preparation from supernatants obtained from one or more non-human cells,
Step 2. purifying the Zika virus preparation;
Step 3. inactivating the virus preparation;
Step 4. transferring the Zika virus preparation into a pharmaceutically acceptable buffer to obtain the inactivated virus composition.

67. A method of preparing a liquid vaccine, the method comprising the following steps:
Step 1. providing the inactivated virus composition of claims 1 to 45,
Step 2. adding an adjuvant preferably an aluminum salt and optionally a further pharmaceutically acceptable buffered liquid to the inactivated virus composition.

68. The method of claim 67, wherein in step 2 the further pharmaceutically acceptable buffered liquid comprises the same buffer as the buffer with the highest concentration in the inactivated virus composition.

69. A product obtainable by the method according to any one of claims 66 to 68.


SEQUENCE LISTING (27 sequences)

SEQ ID NO: 1 - protein, 351 aa, Zika virus
DVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVT

SEQ ID NO: 2 - DNA, 10675 bp, Zika virus
gttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaacaataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccaccaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttctgacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccgagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtatttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggatgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaacgggagttatgttagtgccatcacccaagggaggagggaggaagagactcctgttgagtgcttcgagccctcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggcatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggctctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgtcgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgcaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagagga

SEQ ID NO: 3 - DNA, 20 bp, artificial
  oligo 1 (CpG 1826)
tccatgacgttcctgacgtt

SEQ ID NO: 4 - DNA, 18 bp, artificial
  oligo 2 (CpG 1758)
tctcccagcgtgcgccat

SEQ ID NO: 5 - DNA, 30 bp, artificial
  oligo 3
accgatgacgtcgccggtgacggcaccacg

SEQ ID NO: 6 - DNA, 24 bp, artificial
  oligo 4 (CpG 2006)
tcgtcgttttgtcgttttgtcgtt

SEQ ID NO: 7 - DNA, 20 bp, artificial
  oligo 5 (CpG 1668)
tccatgacgttcctgatgct

SEQ ID NO: 8 - protein, 31 aa, Zika virus
FTKIPAETLHGTVTVEVQYAGTDGPCKVPAQ

SEQ ID NO: 9 - protein, 31 aa, Zika virus; identical to SEQ ID NO: 8

SEQ ID NO: 10 - protein, 31 aa, West Nile Virus
FLGTPADTGHGTVVLELQYTGTDGPCKVPIS

SEQ ID NO: 11 - protein, 31 aa, Japanese Encephalitis Virus
FAKNPADTGHGTVVIELTYSGSDGPCKIPIV

SEQ ID NO: 12 - protein, 31 aa, Saint Louis Encephalitis Virus
FSKNPADTGHGTVIVELQYTGSNGPCRVPIS

SEQ ID NO: 13 - protein, 30 aa, Yellow Fever Virus
FVKNPTDTGHGTVVMQVKVSKGAPCRIPVI

SEQ ID NO: 14 - protein, 31 aa, Dengue virus
LEKEVAETQHGTVLVQVKYEGTDAPCKIPFS

SEQ ID NO: 15 - protein, 31 aa, Dengue virus
VVKEIAETQHGTIVIRVQYEGDGSPCKIPFE

SEQ ID NO: 16 - protein, 31 aa, Dengue virus
LKKEVSETQHGTILIKVEYKGEDAPCKIPFS

SEQ ID NO: 17 - protein, 31 aa, Dengue virus
IDKEMAETQHGTTVVKVKYEGAGAPCKVPIE

SEQ ID NO: 18 - protein, 29 aa, Zika virus
SVKNPMGRGPQRLPVPVNELPHGWKAWGK

SEQ ID NO: 19 - protein, 29 aa, Zika virus
SVKNPMWRGPQRLPVPVNELPHGWKAWGK

SEQ ID NO: 20 - protein, 29 aa, West Nile Virus
KQEGMYKSAPKRLTATTEKLEIGWKAWGK

SEQ ID NO: 21 - protein, 29 aa, Japanese Encephalitis Virus
KPVGRYRSAPKRLSMTQEKFEMGWKAWGK

SEQ ID NO: 22 - protein, 29 aa, Saint Louis Encephalitis Virus
EDPKYYKRAPRRLKKLEDELNYGWKAWGK

SEQ ID NO: 23 - protein, 29 aa, Yellow Fever Virus
DPKNVYQRGTHPFSRIRDGLQYGWKTWGK

SEQ ID NO: 24 - protein, 29 aa, Dengue virus
DVSGILAQGKKMIRPQPMEHKYSWKSWGK

SEQ ID NO: 25 - protein, 29 aa, Dengue virus
DIKGIMQAGKRSLRPQPTELKYSWKTWGK

SEQ ID NO: 26 - protein, 29 aa, Dengue virus
DITGVLEQGKRTLTPQPMELKYSWKTWGK

SEQ ID NO: 27 - protein, 29 aa, Dengue virus
DVKGVLTKGKRALTPPVNDLKYSWKTWGK